(19) United States
(12) Patent Application Publication
(10) Pub. No.: US 2018/0021178 A1
(43) Pub. Date: Jan. 25, 2018

(54) Reduced-Pressure Systems, Dressings, Pump Assemblies And Methods

(71) Applicant: KCI Licensing, Inc., San Antonio, TX (US)

(72) Inventors: Christopher Brian LOCKE, Bournemouth (GB); Richard Daniel John COULTHARD, Verwood (GB); Justin Alexander LONG, Bournemouth (GB)

(21) Appl. No.: 15/666,183

(22) Filed: Aug. 1, 2017

Related U.S. Application Data
(63) Continuation-in-part of application No. 14/386,444, filed on Sep. 19, 2014.
(60) Provisional application No. 61/616,901, filed on Mar. 28, 2012.

(51) Int. Cl. A61F 13/02; A61M 1/00

(52) U.S. Cl. CPC A61F 13/0206; A61M 1/009; A61F 2013/00174

(57) ABSTRACT

According to an illustrative embodiment, a reduced-pressure wound treatment system includes a dressing and a pump assembly. Another illustrative embodiment includes a reduced-wound pressure treatment system including a dressing with a rigid outlet coupled to a manifold, and a pump assembly including a pump and a rigid inlet, the inlet of the pump assembly being removeably connectable to the outlet of the dressing by rigid interlocking formations. A further illustrative embodiment of a reduced-pressure wound treatment system includes a dressing with an outlet, and a pump assembly with an inlet, where the dressing is removeably coupled to the pump assembly solely by the inlet being attached to the outlet, and the dressing cover and pump cover contact each other while the inlet and the outlet are attached together. According to a further illustrative embodiment, a reduced-pressure wound treatment system employs an inductive charger operably electrically charging a pump battery.

### CROSS-REFERENCE TO RELATED APPLICATIONS

This application is a continuation-in-part of U.S. Ser. No. 14/386,444, filed on Sep. 19, 1014, which is a national phase entry of PCT/US2013/034472, filed on Mar. 28, 2013, which claims the benefit of U.S. provisional application Ser. No. 61/616,901, filed Mar. 28, 2012, all of which are incorporated by reference herein.

### FIELD

The present disclosure relates generally to medical treatment systems and, more particularly, but not by way of limitation, to reduced-pressure wound systems, dressings, pump assemblies and methods.

### BACKGROUND

Clinical studies and practice have shown that providing a reduced pressure in proximity to a tissue site augments and accelerates the growth of new tissue at the tissue site. The applications of this phenomenon are numerous, but application of reduced pressure has been particularly successful in treating wounds. This treatment (frequently referred to in the medical community as “negative pressure wound therapy,” “reduced pressure therapy,” or “vacuum therapy”) provides a number of benefits, which may include faster healing and increased formulation of granulation tissue. Typically, when applied to open wounds, reduced pressure is applied to tissue through a porous pad or other manifold device of a reduced-pressure wound dressing. The porous pad distributes reduced pressure to the tissue and channels fluids that are drawn from the tissue into the dressing. When the reduced pressure therapy is completed or the reduced-pressure wound dressing is spent, the reduced-pressure wound dressing is removed from the tissue site and discarded.

### SUMMARY

New and useful systems, apparatus, and methods for providing negative-pressure treatment are set forth in the appending claims. Illustrative embodiments are also provided to enable a person skilled in the art to make and use the claimed subject matter.

For example, some embodiments of an apparatus may comprise a low-profile, absorbent dressing with an integrated micro-pump and wireless charging. A covering may enclose and protect the dressing and pump. The dressing and pump may be permanently joined in some embodiments, or may be connected by a coupling to allow assembly and disassembly by an operator. The apparatus may be flexible or otherwise conformable, and the pump may operate at almost undetectable noise levels, which can be worn by a patient to provide discreet reduced-pressure treatment. A portable power source, such as batteries, can provide power to the pump. Secondary cells may be used, and an inductive charging system may be used to charge the cells. For example, a suitable electrical coil may be placed against an outer surface of the apparatus, aligned with a matching coil on a control board within the apparatus. Alignment and fixation may be facilitated by magnets, which can guide application and hold the external coil in position.

Some embodiments of a dressing may include a patient interface layer, a cover, a manifold, and an absorbent core. A perforated silicone gel adhesive may be suitable as a patient interface layer in some examples. Some embodiments of a cover may be made from a polyurethane film coated with an acrylic adhesive. The manifold may be a low-profile layer of foam or a non-woven material, for example, which can also be configured to transmit significant apposition forces. The absorbent core may suitable comprise a superabsorbent textile in some embodiments. Alternatively or additionally, a perforated film layer may be disposed between the absorbent core and the manifold to prevent backflow of liquid from the absorbent core. An aperture on an upper surface of the dressing can allow transmission of negative pressure, and a liquid-blocking filter can block egress of exudate through the aperture. A filter can also provide a viral and bacterial barrier in some embodiments.

In some embodiments, a system for treating a tissue site with reduced pressure may include a dressing and a pump assembly. The dressing may include a manifold, an absorbent layer, and a cover. The manifold can be adapted to deliver reduced pressure to a tissue site, and the absorbent layer can be in fluid communication with the manifold to absorb liquid from at least one of the manifold layer and the tissue site. The cover may be positioned over the absorbent layer and the manifold to maintain the reduced pressure at the tissue site. The pump assembly may be adapted to provide fluid communication to the tissue site through at least one of the absorbent layer and the manifold of the dressing. In some embodiments, the pump assembly may include a pump and a fluid inlet coupled to the pump, and the fluid inlet may be configured to be removeably and directly connectable to the dressing by interlocking formations.

Other illustrative embodiments of a system for providing reduced-pressure treatment may include an elongated dressing and a pump enclosed in an electronics pouch. The dressing may include an absorbent layer, a manifold, and a dressing cover enclosing the absorbent layer and the manifold. An outlet coupled to the manifold may be accessible through an aperture in the dressing cover. An elongated and flexible cushion may protect the pump in the pouch, and a pump cover may enclose the pump and the cushion. An inlet coupled to the pump may be accessible through an aperture in the pump cover. In some embodiments, the dressing may be removeably coupled to the electronics pouch solely by the inlet being attached to the outlet. The dressing cover and the pump cover may contact each other while the inlet and the outlet are attached.

In some embodiments, a system for reduced-pressure treatment may include a pump, an electrical circuit connected to the pump, a battery connected to the electrical circuit, a flexible cushion, and a flexible pump cover substantially surrounding the pump, the electrical circuit, the battery, and the cushion. The cushion may be located between the electrical circuit and a portion of the pump cover. A dressing interface may be fluidly coupled to the pump. In some embodiments, the system may additionally include a dressing, which may have a rigid outlet. Interlocking formations may removeably couple the dressing to the dressing interface through a rotational movement.

Some illustrative embodiments of a reduced-pressure treatment system may include a dressing with a rigid outlet coupled to a manifold, and a pump assembly including a pump and a rigid inlet, the inlet of the pump assembly being removeably connectable to the outlet of the dressing by rigid interlocking formations. Further illustrative embodiments of a reduced-pressure wound treatment system may include a dressing with an outlet, and a pump assembly with an inlet, where the dressing is removeably coupled to the pump assembly solely by the inlet being attached to the outlet, and the dressing cover and pump cover contact each other while the inlet and the outlet are attached together.

Moreover, some illustrative embodiments of a reduced-pressure treatment system can include a pump assembly including a pump, and electrical circuit, a battery, a flexible cushion and a flexible pump cover, where the cushion is located between the circuit and battery on one hand, and an outer portion of the pump cover on the other hand. Additional illustrative embodiments of a reduced-pressure wound treatment system may include a pump assembly including a flexible cushion surrounding a periphery of a pump. Still other illustrative embodiments of a reduced-pressure treatment system may include a pump, an electrical circuit board connected to the pump, a battery flexibly coupled to the electrical circuit board, and a flexible cover surrounding the pump, the electrical circuit board and the battery.

According to further illustrative embodiments, a reduced-pressure treatment system can employ an inductive charger operably to electrically charge a pump battery while the battery remains within a pump cover.

An illustrative method of use may include electrically charging a pump battery via inductance by an electrical charger for a reduced-pressure wound treatment system. Moreover, additional illustrative method may interlock together an inlet of a pump assembly to an outlet of a dressing through rotation.

The example embodiments described herein may provide significant advantages. For example, some embodiments can provide easy to use, fast and secure interlocking connections between a pump assembly and a dressing. Interlocking connections can be quickly disengaged for pump removal without the need for tools or specialized expertise. Some embodiments may additionally or alternatively provide a thin packaging of the pump assembly and dressing, including fluid interconnections therebetween. Moreover, inductive charging can allow for much easier recharging of an internal pump battery without requiring removal of the battery in some embodiments. Some aspects may also provide an advantageous multifunctional feature employing one or more magnets which can act as an inductive antenna and/or a removable coupling between an electrical charger base and a receiver attached to the pump assembly.

Other features, advantages, and a preferred mode of making and using the claimed subject matter may be understood best with reference to the accompanying drawings and detailed description that follows.

### BRIEF DESCRIPTION OF THE DRAWINGS

FIG. 1 is a side, cross-section view of an illustrative embodiment of a system for treating a tissue site with reduced pressure, including a reduced-pressure dressing coupled to the tissue site;

FIG. 2 is a side, cross-section view of the illustrative reduced-pressure dressing of FIG. 1, including a removable coupling between an electronics pouch and an absorbent pouch of the reduced-pressure dressing;

FIG. 2A is a detail view of a portion of the reduced-pressure dressing that includes a perforation;

FIG. 3 is a top view of the reduced-pressure dressing;

FIG. 4 is a side, cross-section view of the reduced-pressure dressing that shows the electronics pouch being separated from the absorbent pouch;

FIG. 5 is a side, cross-section view showing another illustrative reduced-pressure dressing having a removable coupling between the electronics pouch and absorbent pouch of the reduced-pressure dressing;

FIG. 5A is a detail view of a portion of the reduced-pressure dressing of FIG. 5;

FIG. 6A is a side, cross-section view of an illustrative embodiment of a reduced-pressure dressing having an intermediate cover member and a sealing member that comprises a first sealing member connector and a second sealing member connector;

FIG. 6B is a detail, cross-section view of the reduced-pressure dressing of FIG. 6 in an exploded state;

FIG. 7 is a side, cross-section view of an illustrative reduced-pressure dressing having an intermediate cover member that comprises a first cover connector and a second cover connector;

FIG. 8A is a top view of an illustrative embodiment of a reduced-pressure dressing having an arcuate shape;

FIG. 8B is a perspective view showing the electronics pouch of the reduced-pressure dressing of FIG. 8A being separated from the absorbent pouch along a perforation;

FIG. 9 is an exploded, perspective view of an illustrative embodiment of a reduced pressure dressing having first envelope that is removably coupled to a second envelope;

FIG. 10 is a perspective view showing another illustrative embodiment of a reduced-pressure wound system, with a pump assembly coupled to a dressing;

FIG. 11 is a perspective view showing the FIG. 10 system, but with the pump assembly rotated to an uncoupled position;

FIG. 12 is a perspective view showing the FIG. 10 system, but with the pump assembly uncoupled and inverted;

FIG. 13 is a cross-sectional view, taken along line 13-13 of FIG. 10, showing the pump assembly coupled to the dressing and placed on a patient's tissue;

FIG. 14 is a top exploded perspective view showing the pump assembly and a portion of the dressing of the FIG. 10 system;

FIG. 15 is a bottom exploded perspective view showing the pump assembly and a portion of the dressing of the FIG. 10 system;

FIG. 16 is a top perspective view showing the pump assembly of the FIG. 10 system, with an outer cover portion removed;

FIG. 17 is a bottom perspective view, taken opposite that of FIG. 16, showing the pump assembly, with a bottom cover portion removed;

FIG. 18 is a fragmentary top perspective view showing the pump assembly and a portion of the dressing of the FIG. 10 system;

FIG. 19 is a partially exploded top perspective view of the FIG. 10 system, with various pump assembly and dressing components removed;

FIG. 20 is a schematic section view of another example of a dressing that may be associated with the system of FIG. 10;

FIG. 21 is a detail view illustrating additional features that may be associated with some examples of the dressing of FIG. 20;

FIG. 22 is a partially fragmentary side elevational view showing a charger coupled to the pump assembly and to a portion of the dressing in another illustrative embodiment of a reduced-pressure wound system;

FIG. 23 is a partially exploded top perspective view of the charger and pump assembly of the FIG. 22 system;

FIG. 24 is a partially exploded bottom perspective view showing the charger and pump assembly of the FIG. 22 system;

FIG. 25 is a diagrammatic perspective view showing portions of the charger of the FIG. 22 system;

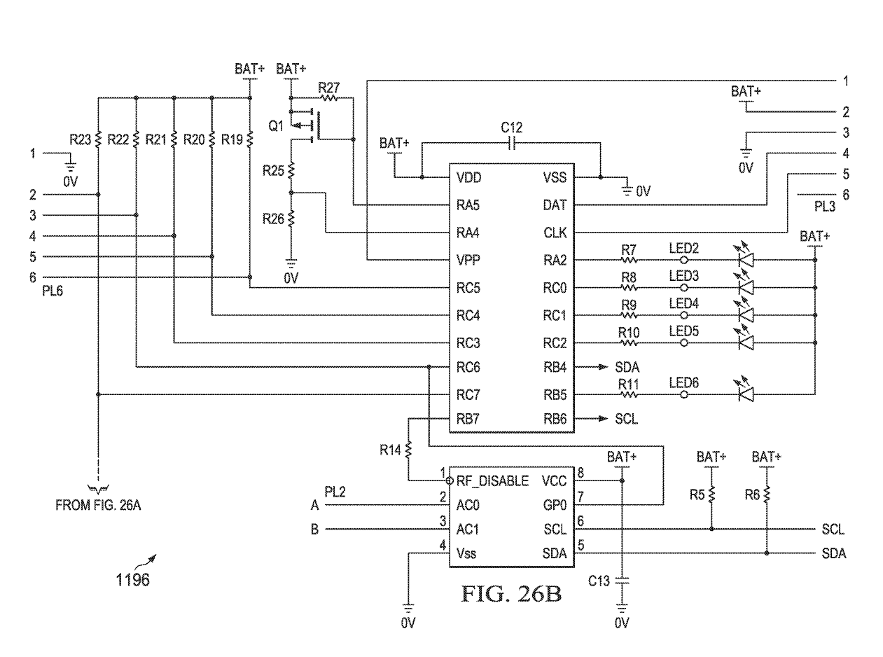

FIGS. 26A, 26B and 27 are circuit diagrams showing the charger of the FIG. 22 system;

FIG. 28 is a top elevational view showing another illustrative embodiment of a reduced-pressure wound system with the pump assembly coupled to a bridge of the dressing;

FIG. 29 is a cross-sectional view, taken along line 29-29 of FIG. 28, showing the bridge portion of the dressing; and

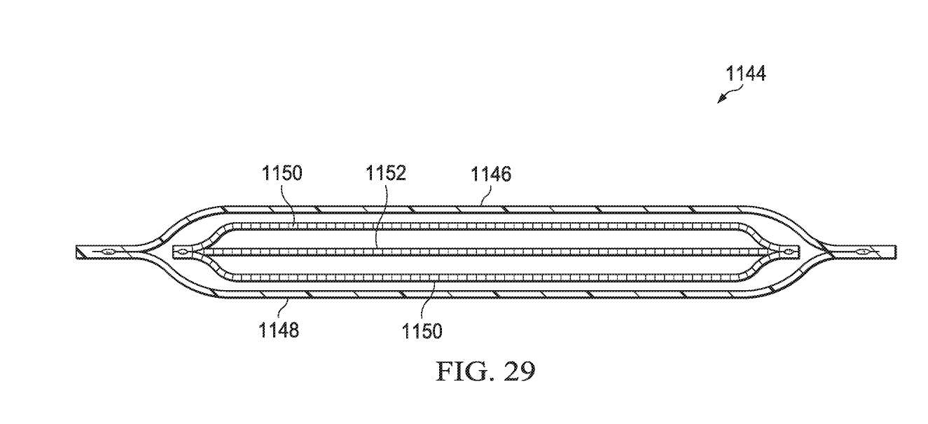

FIG. 30 is a top exploded perspective view showing the bridge portion of the dressing of the FIG. 28 system.

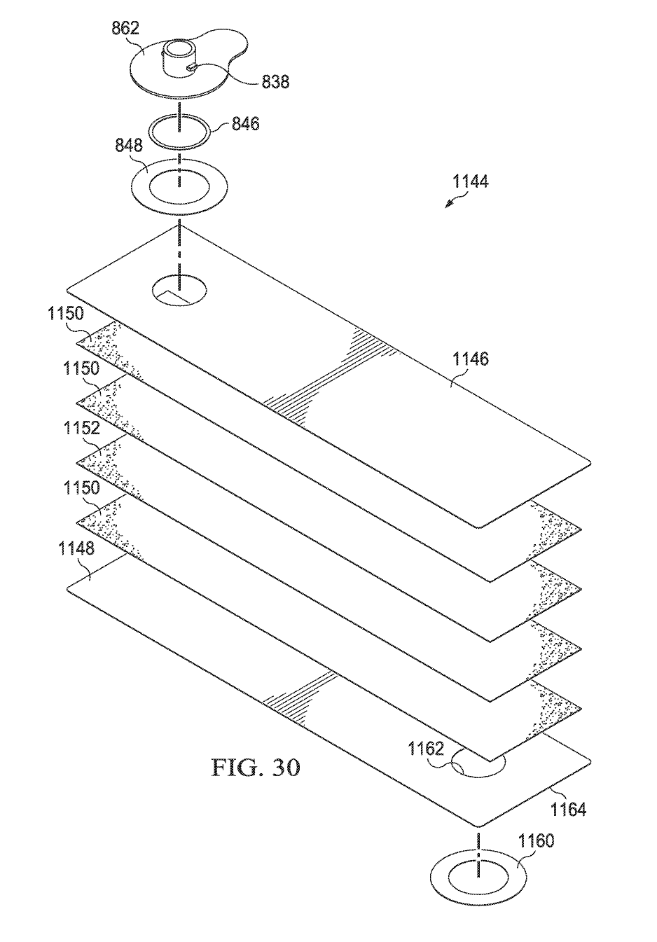

### DETAILED DESCRIPTION OF ILLUSTRATIVE EMBODIMENTS

In the following detailed description of the illustrative, non-limiting embodiments, reference is made to the accompanying drawings that form a part hereof. These illustrative embodiments are described in sufficient detail to enable those skilled in the art to practice the invention. It is understood that other embodiments may be utilized and that logical structural, mechanical, electrical, and chemical changes may be made without departing from the spirit or scope of the invention. To avoid detail not necessary to enable those skilled in the art to practice the embodiments described herein, the description may omit certain information known to those skilled in the art. The following detailed description is not to be taken in a limiting sense, and the scope of the illustrative embodiments is defined only by the appended claims.

Wound dressings composed of traditional dressing materials typically do not contain electronic components. Yet recent and more advanced wound dressings include electronic components to deliver therapy to wounds and to monitor conditions at wound sites. This may pose a difficultly when the dressing has been used and the time comes to dispose of the dressing. Used wound dressings that include biological or clinical waste are frequently required by law to be disposed by approved methods. For example, regulations may require the incineration of clinical waste to limit the risk of spreading disease. Similarly, the disposal of electronic components is also regulated by law in many jurisdictions. Such regulations may require that used electronic components be disassembled and recycled, or sent to a specific waste handling center that is equipped to dispose of electronic components with minimal environmental impact. The approved methods for disposing of clinical waste and electronic waste, however, are normally not compatible with one another. Thus, in the case of a used wound dressing that includes electronic components, the electronic components may be separated from the clinical waste prior to disposal. After separation, the clinical waste portion and electronic waste portions of the spent wound dressing may be sent to different facilities for disposal. Depending on the configuration of the wound dressing, however, separating the electronics from the remainder of the wound dressing may be messy, impractical, and unsanitary.

The illustrative embodiments include a wound dressing that functions as a single unit to treat a wound but allows for the separation of the electronic components from components that have absorbed clinical waste prior to disposal. Such a wound dressing allows the appropriate disposal of the clinical waste and recycling of electronic components. The illustrative embodiments also include wound dressing components that may be recombined to enable a wound dressing to stay in place while electronic components, such as batteries, are replaced to extend the life of the wound dressing.

The illustrative embodiments provide a reduced-pressure wound dressing having a reliable seal between dressing components that can be broken apart without exposing a user or caregiver to unnecessary contact with fluids absorbed by the dressing. The reduced-pressure wound dressing allows for easy and appropriate disposal of the components depending on the type of waste (e.g., as clinical waste or electronic waste). In addition, the illustrative embodiments provide an integrated wound dressing and reduced-pressure source (i.e., a pump) that may be manufactured either as a single unit or as separate modules. Parts of a modular system may be manufactured in separate facilities and different sterilization processes may be employed to different components of the system. For example, portions of the dressing that include electronic components may be sterilized using Ethylene Oxide, Super Critical Carbon Dioxide, or other sterilization methods that do not degrade the electronics. Other portions of the dressing may be sterilized using other methods, such as Gamma Irradiation or E-Beam sterilization, dependent on material compatibility. An illustrative reduced-pressure dressing alleviates the need for a remote reduced-pressure source or therapy unit that is connected via a tube or conduit, as used by more typical dressings that provide reduced pressure to a tissue site. The illustrative reduced-pressure dressing is a self-contained dressing or therapy unit that can be separated on disposal with minimal user intervention and effort.

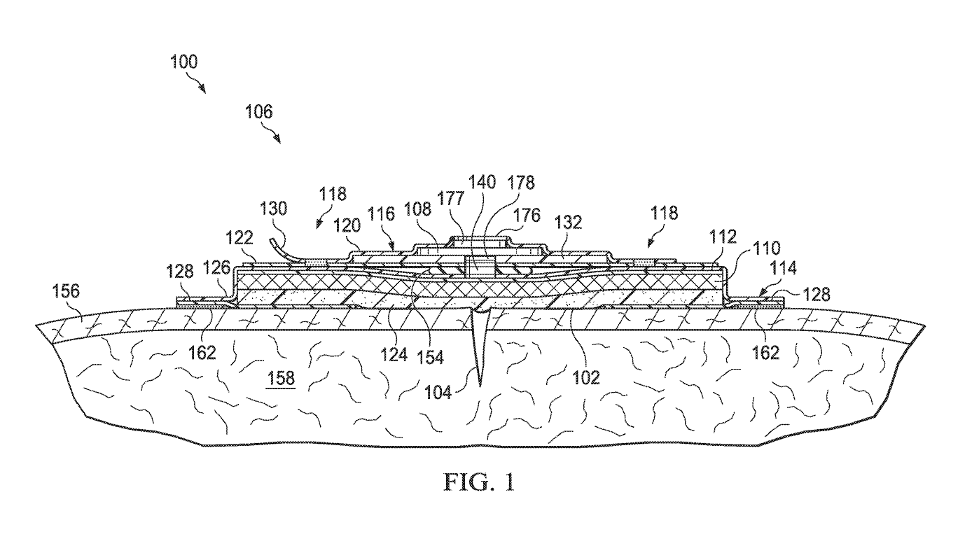

In one embodiment, an absorbent, reduced-pressure dressing has an onboard reduced-pressure source, control system, and power source. Referring now to the drawings and initially to FIG. 1, an illustrative embodiment of a system 100 for treating a tissue site 102, e.g., a wound 104 or a cavity, with reduced pressure is presented. The tissue site 102 may be, for example, the wound 104 extending through epidermis 156 and into subcutaneous tissue 158, or any other tissue site. Reduced pressure generally refers to a pressure less than the ambient pressure at a tissue site that is being subjected to treatment. In most cases, this reduced pressure will be less than the atmospheric pressure at which the patient is located. Alternatively, the reduced pressure may be less than a hydrostatic pressure at the tissue site 102. Unless otherwise indicated, values of pressure stated herein are gauge pressures. The reduced pressure delivered may be constant or varied (patterned or random) and may be delivered continuously or intermittently. Consistent with the use herein, unless otherwise indicated, an increase in reduced pressure or vacuum pressure typically refers to a relative reduction in absolute pressure.

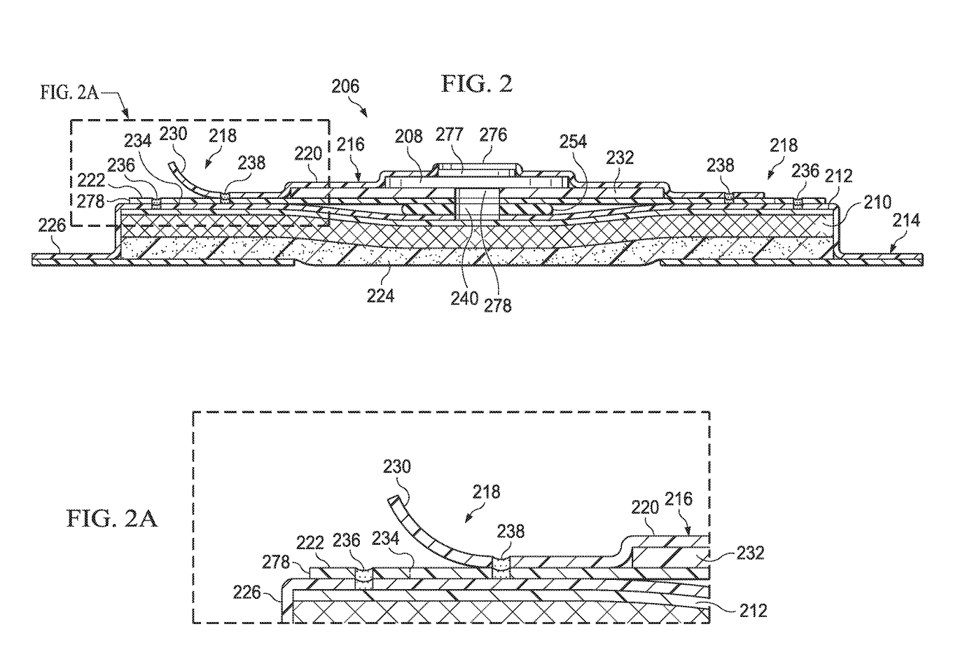

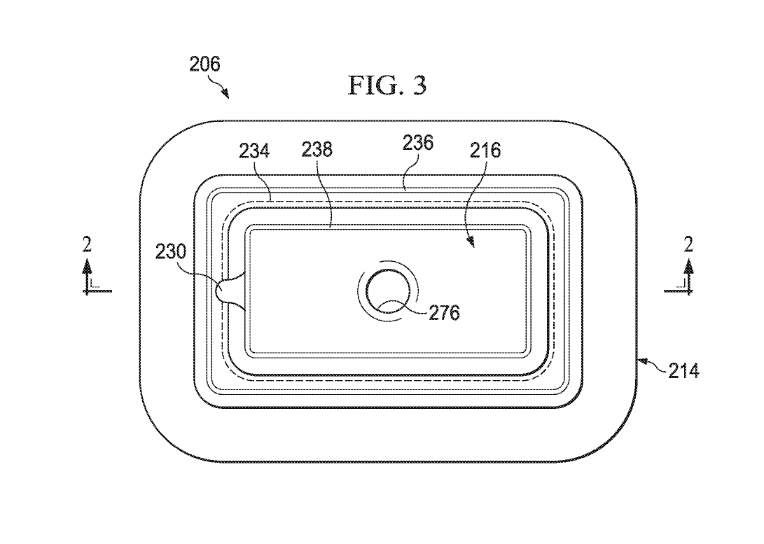

The system 100 includes a reduced-pressure dressing 106 for disposing proximate to the tissue site 102. The reduced-pressure dressing 106 includes absorbent materials and has the ability to deliver reduced pressure to the tissue site 102. The reduced-pressure dressing 106 includes an absorbent pouch 114 fluidly sealed and mechanically connected, or coupled, to an electronics pouch 116 by a removable coupling 118 or a sealing member 154 that pneumatically connects the pouches. As used herein, the word “or” is not mutually exclusive. The electronics pouch 116 and absorbent pouch 114 are joined together such that there is a secure bond between the pouches. The secure bond may be a high-frequency weld around the periphery of the electronics pouch 116. FIGS. 2-9 show similar systems, and variation is shown between figures in order to show some of the potential variations in the illustrative system 100.

The system 100 may be used with various different types of tissue sites 102. The tissue site 102 may be the bodily tissue of any human, animal, or other organism, including bone tissue, adipose tissue, muscle tissue, dermal tissue, vascular tissue, connective tissue, cartilage, tendons, ligaments, body cavity or any other tissue. Treatment of the tissue site 102 may include removal of fluids, e.g., exudate or ascites.

Referring again to FIG. 1, the electronics pouch 116 of the reduced-pressure dressing 106 is formed by coupling a first electronics cover 120 to a second electronics cover 122, wherein the second electronics cover 122 is on the patient-facing side of the electronics pouch 116. In one embodiment, one or more sub parts, e.g., sheets of elastomeric film, form the first electronics cover 120 and the second electronics cover 122. The electronics pouch 116 may also be formed by other techniques such as casting or molding the electronics pouch 116 from a polymer. The electronics pouch 116, or pump pouch, of FIG. 1 includes a pump 108. Within the electronics pouch 116, the pump 108 is mounted to a substrate 132 that is formed from a printed circuit board material such as polyimide, phenolic or another suitable material. The electronics pouch may also include a processor, a power source, and a communication system (not shown) that control the pump 108, power the pump 108, and transmit and receive data. In use, the pump 108 delivers reduced-pressure to the absorbent pouch 114 through an aperture 178 in the substrate 132 that is coupled to the second electronics cover 122. The first electronics cover 120 of the electronics pouch 116 includes a vent 176 to fluidly couple an exhaust from the pump 108 to an exterior of the reduced-pressure dressing 106. An odor filter 177 may be installed within the vent 176 to prevent the reduced-pressure dressing 106 from emitting odor from the wound 104.

The pump 108 may be a micro-pump device and may take numerous forms, such as a piezoelectric pump, peristaltic pump, or other miniaturized pump. In one embodiment, the pump 108 is an acoustic resonance pump that applies the principle of acoustic resonance to generate pressure oscillations within a cavity and motivate fluid through the pump 108. The pump 108 may be the type of micro-pump shown in United States Patent Publication 2009/0240185 (application Ser. No. 12/398,904; filed 5 Mar. 2009), entitled, “Dressing and Method for Applying Reduced Pressure To and Collecting And Storing Fluid from a Tissue Site,” which is incorporated herein for all purposes.

The pump 108 is small and light enough to allow the reduced-pressure dressing 106 to be maintained on the tissue site 102 without causing discomfort to the patient. The size and weight of the micro-pump may be such that the reduced-pressure dressing 106 does not pull or otherwise adversely affect the tissue site 102. In one illustrative embodiment, the micro-pump may be a disc pump having a piezoelectric actuator similar to that previously described. Reference is also made to the pumps shown in United States Patent Publication 2009/0087323 and United States Patent Publication 2009/0240185, which are hereby incorporated by reference for all purposes. It should be understood that alternative pump technologies may be utilized and that rotary, linear, or other configurations of pumps may be utilized.

The pump 108 has sufficient flow, reduced pressure, and operation life characteristics to enable continuous application of reduced pressure treatment. The flow may range between about 5-1200 ml/min and the reduced pressure may range between about −50 and −200 mm Hg (−6.6 to −26.6 kPa). It should be understood that alternative ranges may be utilized depending on the configuration of the reduced-pressure dressing 106, size of wound, or type of wound. In one illustrative embodiment, multiple pumps may be positioned in a single dressing to deliver increased flow rates or vacuum levels as required.

In use, the pump 108 generates reduced pressure that is delivered to the tissue site 102 via the absorbent pouch 114. To deliver reduced-pressure to the tissue site 102, the pump 108 applies reduced-pressure through the aperture 178 in the substrate 132 or an aperture in the pump base if no substrate 132 is present. In the embodiment of FIG. 1, a sealing member 154 having a sealing member aperture 140 fluidly couples the electronics pouch 116 to the absorbent pouch 114. The sealing member 154 provides a fluid seal by coupling to, for example, the substrate 132 of the electronics pouch 116 and the absorbent pouch 114. In other embodiments, the reduced-pressure dressing 106 omits the sealing member 154 and the electronics pouch 116 and absorbent pouch 114 are fluidly coupled by a direct coupling. When applying reduced-pressure to the tissue site 102, the absorbent pouch 114 may receive and retain fluids from the tissue site 102.

In one embodiment, the sealing member 154 is a sealing ring that provides a pneumatic seal between the pump 108 and the absorbent pouch 114. One side of the sealing ring may be bonded to the substrate 132 to which the pump 108 is mounted and the other side of the sealing ring may be bonded to the absorbent pouch 114.

The absorbent pouch 114 applies reduced pressure from the pump 108 to the tissue site 102. The absorbent pouch 114 includes a manifold layer 124 formed from a manifold material and is applied adjacent to the tissue site 102 to distribute reduced pressure. Generally, a manifold is a substance or structure that assists in applying reduced pressure to, delivering fluids to, or removing fluids from a tissue site 102. The manifold layer 124 typically includes a plurality of flow channels or pathways that distribute fluids provided to and removed from the tissue site 102 around the manifold layer 124. In one illustrative embodiment, the flow channels or pathways are interconnected to improve distribution of fluids provided to or removed from the tissue site 102. The manifold layer 124 may be a biocompatible material that is capable of being placed in contact with the tissue site 102 and distributing reduced pressure to the tissue site 102. Examples of materials used to form the manifold layer 124 may include without limitation the following: materials that have structural elements arranged to form flow channels, e.g., cellular foam, open-cell foam, porous tissue collections, liquids, gels, and foams that include, or cure to include, flow channels; foam; gauze; felted mat; or any other material suited to a particular biological application.

In one embodiment, the manifold layer 124 is a porous foam and includes a plurality of interconnected cells or pores that act as flow channels. The porous foam may be a polyurethane, open-cell foam such as GRANUFOAM™ dressing available from Kinetic Concepts, Incorporated of San Antonio, Tex. In some situations, the manifold layer 124 may also be used to distribute fluids such as medications, antibacterials, growth factors, and various solutions to the tissue site 102. Other layers may be included in or on manifold layer 124, such as absorptive materials, wicking materials, hydrophobic materials, and hydrophilic materials.

In one embodiment, the manifold layer 124 distributes reduced pressure generated by the pump 108 and may draw exudate from the wound 104. To retain the exudate, the manifold layer 124 is coupled to an absorbent layer 110 that functions to receive and retain fluids such as exudate from the tissue site 102. The absorbent layer 110 may be made from any material capable of absorbing liquid. For example, the absorbent layer 110 may be made from super absorbent fibers. The super absorbent fibers may retain or bond to the liquid in conjunction with a physical or chemical change to the fibers. In one non-limiting example, the super absorbent fiber may include the Super Absorbent Fiber (SAF) material from Technical Absorbents, Ltd. of Grimsby, United Kingdom. The absorbent layer 110 may be a sheet or mat of fibrous material in which the fibers absorb liquid from the tissue site 102. The structure of the absorbent layer 110 that contains the fibers may be either woven or non-woven. The fibers in the absorbent layer 110 may gel upon contact with the liquid, thereby trapping the liquid. Spaces or voids between the fibers may allow reduced pressure that is applied to the absorbent layer 110 to be transferred within and through the absorbent layer 110.

To prevent liquid (e.g., exudate) from escaping the absorbent pouch 114 and entering the electronics pouch 116, a liquid-air separator 112, e.g., a hydrophobic filter, may be placed between absorbent layer 110 and a first cover of the absorbent pouch 114. In such an embodiment, the first cover 126 of the absorbent pouch 114 is coupled about the perimeter of the sealing member 154 to form a fluid seal.

In an embodiment, an intermediate manifold may be applied between the reduced-pressure dressing 106 and a portion of the tissue site 102. The intermediate manifold may be constructed from bioresorbable materials that may remain in a patient's body following use of the reduced-pressure dressing 106. Suitable bioresorbable materials may include, without limitation, a polymeric blend of polylactic acid (PLA) and polyglycolic acid (PGA). The polymeric blend may also include without limitation polycarbonates, polyfumarates, and capralactones. The intermediate manifold may further serve as a scaffold for new cell-growth, or a scaffold material may be used in conjunction with the intermediate manifold to promote cell-growth. A scaffold is a substance or structure used to enhance or promote the growth of cells or formation of tissue, such as a three-dimensional porous structure that provides a template for cell growth. Illustrative examples of scaffold materials include calcium phosphate, collagen, PLA/PGA, coral hydroxy apatites, carbonates, or processed allograft materials. In an embodiment, the reduced-pressure dressing 106 also includes an interface layer, or comfort layer, for placing between the tissue site 102 and the manifold layer 124.

The absorbent pouch 114 maintains a fluid coupling with the tissue site 102 to apply reduced-pressure. As such, the perimeter of the absorbent pouch 114 may be coupled to the tissue site 102 to form a sealed space. This coupling creates a fluid seal around the tissue site 102 that may be achieved by coupling the first cover 126 of the absorbent pouch 114 to the tissue site 102 using an attachment device. In such an embodiment, the first cover 126 is coupled to the manifold layer 124 or a comfort layer so that the absorbent layer 110 will maintain structural integrity when removed from the tissue site 102. In another embodiment, the first cover 126 is coupled to a second cover 128 in the manner described above with regard to the first electronics cover 120 and second electronics cover 122 of the electronics pouch 116. In an embodiment, the second cover 128 is coupled to the tissue site 102 to create the fluid seal when the reduced-pressure dressing 106 is applied to the tissue site 102. Upon removal of the reduced-pressure dressing 106 from the tissue site 102, the coupling between the first cover 126 and second cover 128 prevents the layers of the absorbent pouch 114 from separating so that the absorbent pouch 114 may be discarded as a unit.

To maintain the fluid seal, the first cover 126 and second cover 128 of the absorbent pouch 114, and the first electronics cover 120 and second electronics cover 122 of the electronics pouch 116 may be formed from an impermeable or semi-permeable, elastomeric material. Elastomeric materials have the properties of an elastomer or, more generally, a polymeric material that has rubber-like properties. More specifically, most elastomers have ultimate elongations greater than 100% and a significant amount of resilience. The resilience of a material refers to the material's ability to recover from an elastic deformation. Examples of elastomers include, but are not limited to, natural rubbers, polyisoprene, styrene butadiene rubber, chloroprene rubber, polybutadiene, nitrile rubber, butyl rubber, ethylene propylene rubber, ethylene propylene diene monomer, chlorosulfonated polyethylene, polysulfide rubber, polyurethane (PU), EVA film, co-polyester, and silicones. Additional, specific examples of dressing sealing member materials include a silicone drape, 3M Tegaderm® drape, polyurethane (PU) drape such as one available from Avery Dennison Corporation of Pasadena, Calif. The reduced-pressure dressing forms a sealed space over the tissue site 102, which may or may not contain the pump 108. The elastomeric material may be a thin, flexible elastomeric film.

An attachment device 162 may be used to couple the first cover 126 or second cover 128 to the patient's epidermis or another intermediate layer, such as a gasket or additional sealing device. The attachment device 162 may take numerous forms. For example, the attachment device 162 may be a medically acceptable, pressure-sensitive adhesive that extends about a periphery or all of the first cover 126 (or second cover 128) or covers at least a portion of a patient-facing side of the reduced-pressure dressing 106 over the epidermis 156.

As noted above, the reduced-pressure dressing 106 includes the removable coupling 118 between the electronics pouch 116 and the absorbent pouch 114. The removable coupling 118 allows a caregiver to separate the electronics pouch 116 from the absorbent pouch 114 by exerting a force on a portion of the electronics pouch 116, such as tab 130. An example of such a removable coupling is described in more detail with regard to FIGS. 2-4.

Turning now to FIGS. 2-4, the reduced-pressure dressing 206 includes a removable coupling 218 that facilitates the separation of the electronics pouch 216 from the absorbent pouch 214 after use. The removable coupling 218 includes a first bond 236 and a second bond 238 offset from the first bond 236. The first bond 236 and second bond 238 may be any suitable type of joining technology, bond or coupling, including a high frequency weld, an ultrasonic weld, a heat weld, an adhesive bond, and a molded part line. In one embodiment, the first bond 236 couples a second electronics cover 222 to a first cover 226 of an absorbent pouch 214. The second bond 238 is offset from the first bond 236 and further from the perimeter 278 of the reduced-pressure dressing 206 than the first bond 236. The first bond 236 should be strong enough so that unintended separation of the electronics pouch 216 from the absorbent pouch 214 does not occur. The first bond 236 may be a weld or other joint that provides a pneumatic seal, but a pneumatic seal between the pouches may instead be provided by another component or weld that is within the boundary of the first bond 236, such as a sealing member 254. A perforation 234 extends through the first electronics cover 220 and second electronics cover 222 between the first bond 236 and second bond 238, i.e., inside of the first bond 236 but outside of the second bond 238. The perforation 234 provides a separation line where the first electronics cover 220 and second electronics cover 222 can be torn to separate the electronics pouch 216 from the absorbent pouch 214. To facilitate separation of the electronics pouch 216 from the absorbent pouch 214, the first electronics cover 220 may include a tab 230 bonded to the first electronics cover 220 using any of the bond types described above, or formed integrally to the first electronics cover 220. Alternatively, the first electronics cover 220 may include a hole that allows a separation force to be exerted on the electronics pouch 216. In one embodiment, pulling the tab 230 causes a tear to develop and propagate along the weakened path of the perforation 234 until the electronics pouch 216 separates from the absorbent pouch 214.

In one embodiment, the first bond 236 couples the second electronics cover 222 to both the first electronics cover 220 and first cover 226. In another embodiment, the first bond 236 couples the first electronics cover 220 to the second electronics cover 222. In such embodiments, the second electronics cover 222 couples to the first cover 226 at any suitable location that is outside of the perforation 234 to preserve the coupling of the electronics pouch 216 to the absorbent pouch 214 until the electronics pouch 216 is torn along the perforation 234.

The dimensions of the perforation 234 are dependent on the material used to manufacture the electronics pouch 216 or absorbent pouch 214 as well as the location of the perforation 234. The perforation 234 should weaken the material so that the strength of the perforated area is significantly less than the tear strength of the pouch material. In an embodiment where the material is Exopack DEV 09-80A or Inspire 70980, the perforation 234 may have the dimensions of 0.1 mm land and between 0.1 mm and 0.5 mm space.

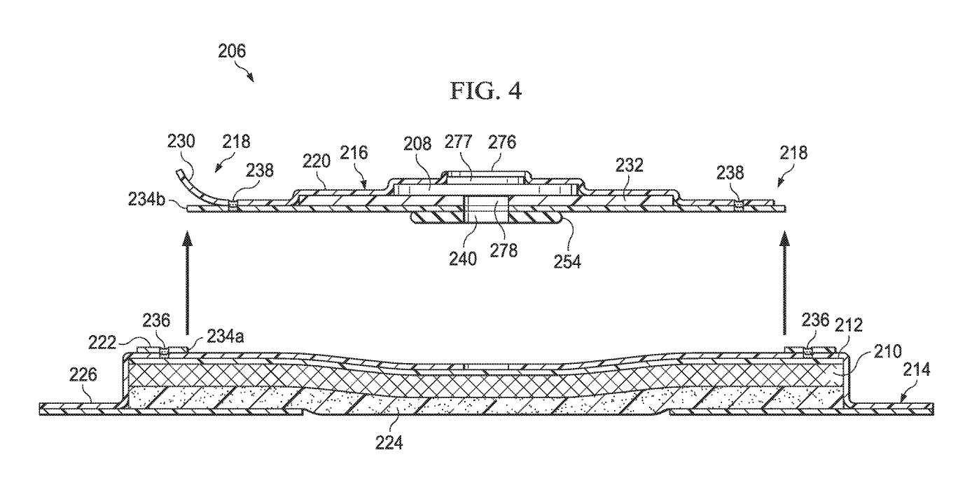

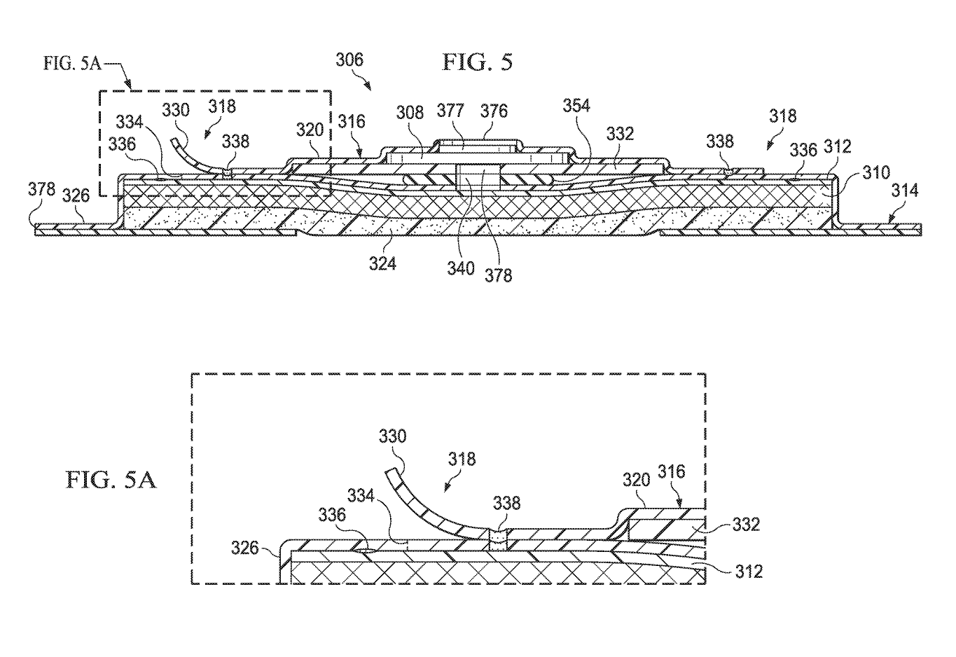

FIG. 3 illustrates a possible arrangement of the first bond 236, perforation 234, and second bond 238 and FIG. 4 shows how the electronics pouch 216 separates from the absorbent pouch 214 after being torn along the perforation 234. When separated, the portion of the reduced-pressure dressing 206 that retains the absorbent pouch 214 has a first perforation line 234a and the electronics pouch 216 has a second perforation line 234b indicating the points of separation. In the illustrative embodiment of FIGS. 2-4, the sealing member 254 is shown as being coupled to the patient-facing side of the electronics pouch 216 and releasably coupled to the absorbent pouch 214. In another embodiment, however, the sealing member 254 is coupled to the absorbent pouch 214 and releasably coupled to the electronics pouch 216.

In an embodiment, the sealing member 254 is a sealing ring, and an adhesive is used to couple the sealing ring to the substrate 232 of the pump 208 or to the first cover 226 of the absorbent pouch 214. The properties of the adhesive applied to the surfaces of the sealing ring may be altered so that when the pouches are separated, the sealing ring remains adhered to either the substrate 232 or the first cover 226. If the sealing ring is attached by welding, the seal ring itself can have a weakened area to facilitate tearing to separate the sealing ring from the electronics pouch 216 or absorbent pouch 214 when the electronics pouch 216 is removed. The sealing ring may then be disposed appropriately. Adhesives that may be used to adhere the sealing ring to the substrate 232 of the first cover 226 may be based on Acrylic Pressure Sensitive Adhesives (PSA), such as 3M 927, or a UV liquid adhesive such as Dymax 1201-M-SC.

The sealing member 254 may be a single flexible material that has adhesive coating on each side to couple to the electronics pouch 216 and absorbent pouch 214. The sealing member 254 also provides a fluid seal between the electronics pouch 216 and absorbent pouch 214. The flexible material may be closed cell foam, such as foam manufactured from neoprene or ethylene-vinyl acetate (EVA). Additionally, the flexible material may provide a level of padding between the electronics pouch 216 and absorbent pouch 214, thereby adding flexibility to the reduced-pressure dressing 206. In an embodiment, the sealing member material may be a solid elastomeric material, such as a thermoplastic elastomer (TPE), or a rigid material. Where an adhesive is used to hold the sealing member 254 in place, the adhesive properties can be altered between the two sides of the sealing member 254 so that on separation, the sealing member 254 remains coupled to either the electronics pouch 216 or the absorbent pouch 214.

In another embodiment, the electronics pouch 216 couples directly to the absorbent pouch 214. In such an embodiment, a portion of the electronics pouch 216 or the absorbent pouch 214 may include a breakaway feature, such as a weakened area in the pouch material or a breakaway feature in the substrate 232 to facilitate separation of the pouches.

Together, FIGS. 2-4 show that a caregiver may separate the electronics pouch 216 from the absorbent pouch 214 by grasping the tab 230 and exerting a force to tear the first electronics cover 220 and second electronics cover 222 around the perimeter of the electronics pouch 216. After generating the tear, the electronics pouch 216 may be grasped and pulled to apply pressure to the sealing member 254, which may be a sealing ring. Once the sealing member 254 is separated from the electronics pouch 216, the electronics pouch 216 is completely free from the absorbent pouch 214 and the pouches may be discarded separately.

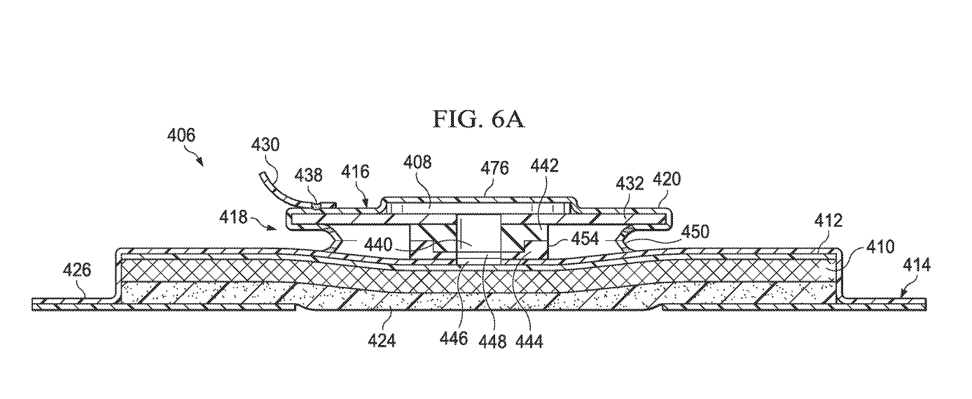

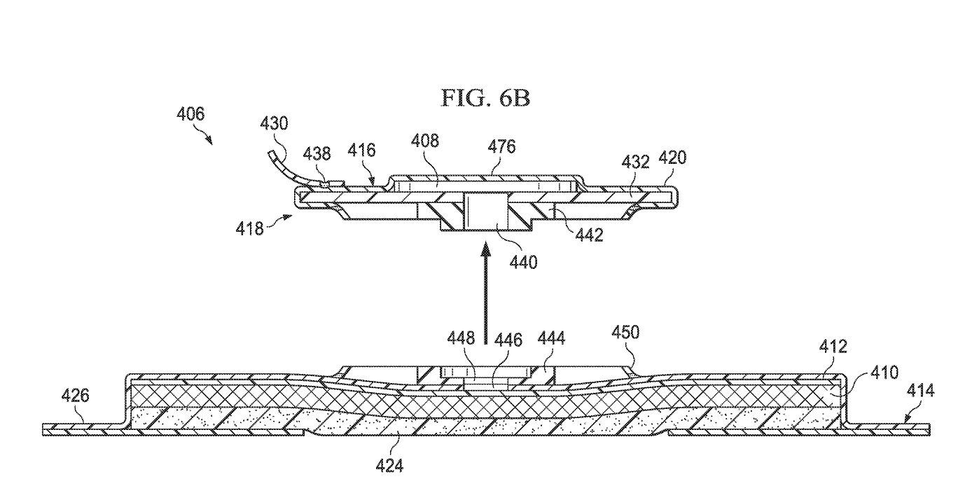

FIG. 5 shows another illustrative embodiment of a reduced-pressure dressing 306 that is similar in many respects to the dressings of FIGS. 1-4 but omits a second electronics cover. In the embodiment, an upper layer of the absorbent pouch 314, such as a liquid-air separator 312, is coupled to the first cover 326 by a first bond 336. Inside of the first bond, the first cover 326 includes a perforation 334. Inside of the perforation 334, the first cover 326 is coupled to the first electronics cover 320 by a second bond 338. In this embodiment, the first cover 326 is also coupled to the substrate 332 that forms a portion of patient-facing side of the electronics pouch 316. Similar to the embodiments of FIGS. 1-4, the reduced-pressure dressing 306 may be torn along the perforation 334 to separate the electronics pouch 316 from the absorbent pouch 314. In this embodiment, the tab 330 may merely be an extension of the first electronics cover 320.

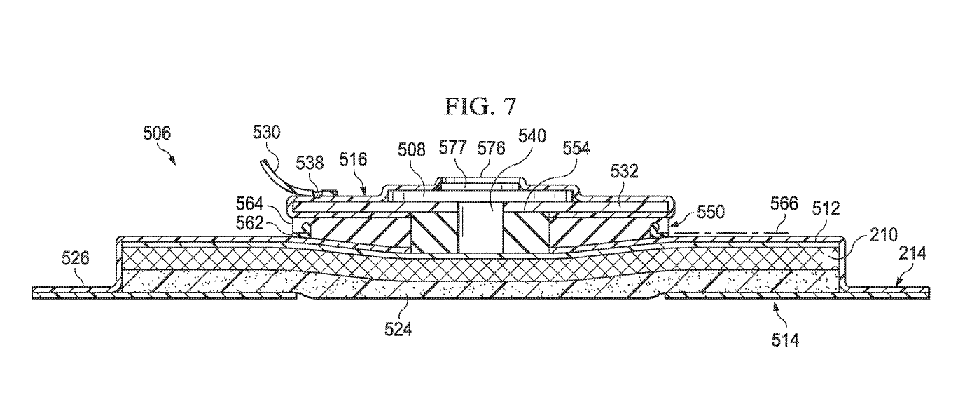

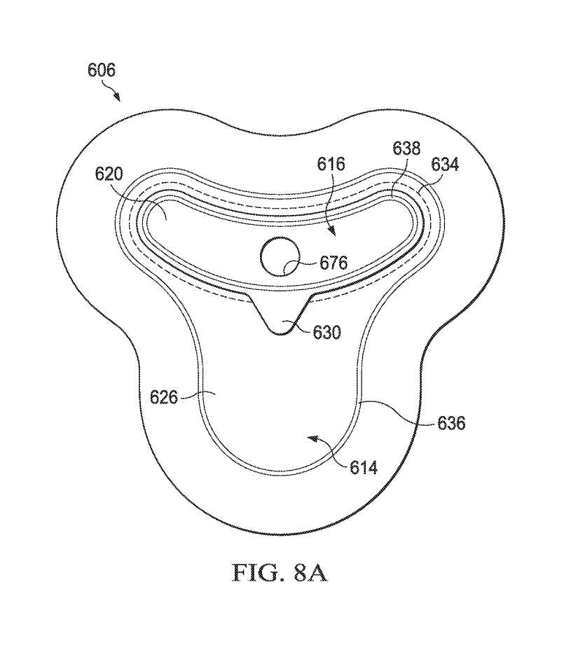

FIGS. 6A and 6B show another illustrative embodiment of a reduced-pressure dressing 406 having an electronics pouch 416 attached to an absorbent pouch 414 by a removable coupling 418. In the embodiment, the first cover 426 of the absorbent pouch 414 is coupled to a proximate side of the sealing member 454. The opposing side of the sealing member 454 is coupled to the second electronics cover 422 or the substrate 432 of the electronics pouch 416. In addition, the first cover 426 and first electronics cover 420 (or second electronics cover 422) are coupled to one another by the removable coupling 418, which is an intermediate cover member 450. The intermediate cover member 450 may include a perforation or be formed of a material that is easier to tear than the material that forms the pouches to facilitate separation of the electronics pouch 416 from the absorbent pouch 414.

In an embodiment, the intermediate cover member 450 provides a fluid seal between the electronics pouch 416 and the absorbent pouch 414, thereby alleviating the need for a sealing member 454. The intermediate cover member 450 may add flexibility between the absorbent pouch 414 and electronics pouch 416 in such an embodiment. The intermediate cover member 450 is bonded to the substrate 432 to which the pump 408 is mounted and bonded or welded to the first cover 426 of the absorbent pouch 414. The material that forms the intermediate cover member 450 is selected such that, when the electronics pouch 416 is separated from the absorbent pouch 414, the intermediate cover member 450 will break before the integrity of either pouch is compromised. In another embodiment, the separation occurs at either the bond between the intermediate cover member 450 and the absorbent pouch 414 or the bond between the intermediate cover member 450 and the electronics pouch 416.

In one embodiment, the sealing member 454 is formed from a first sealing connector 442 coupled to the substrate 432 of the electronics pouch 416 and a second sealing connector 444 coupled to the absorbent pouch 414. The first sealing connector 442 is releasably coupled to the second sealing connector 444. As FIG. 6B shows, the releasable coupling between the first sealing connector 442 and second sealing connector 444 results in the first sealing connector 442 remaining coupled to the electronics pouch 416 and the second sealing connector 444 remaining coupled to the absorbent pouch 414 when the pouches are separated. In one embodiment the sealing member 454 or second sealing connector 444 includes additional elements, such as a liquid-air separator 446 and an odor filter 448. Including the liquid-air separator 446 within the sealing member 454 may alleviate the need for such an element in the absorbent pouch, enabling a smaller part to perform the function of preventing liquids (e.g., exudate) from entering the electronics pouch 416. Similarly, the sealing member 454 may include the odor filter 448, which may be a charcoal filter, thereby alleviating the need to install such an element in another portion of the reduced-pressure dressing 406. The sealing member 454 may be formed from a polymer, such as a polyvinyl chloride (PVC) or acrylonitrile butadiene styrene (ABS) polymer. In an embodiment, the sealing member 454 may instead be formed from polyurethane or another suitable material that is compatible with the pouch cover material and weldable using a high-frequency welding process. In one embodiment, the sealing member 454 couples to the second electronics cover 422 or directly to the substrate 432 using an adhesive.

In one embodiment, a breakable connection piece is securely bonded to both the electronics pouch 416 and absorbent pouch 414 to serve the function of both a sealing member 454 and intermediate cover member 450. In such an embodiment, the pouches may be separated by breaking the breakable connection piece. Such a breakable connection piece may be manufactured from a plastic molding having a weakened breakaway area that causes the breakable connection piece to break in a predictable and controllable manner. The breakable connection piece may be made from injection molded thermoplastic polyurethane (TPU), such as Pellethane® 2363-80AE having a durometer of 80 on the Shore A scale. The thickness of the weakened area may be in the range of 0.05 mm to 0.08 mm, thereby enabling a controlled tear, or break, to be induced without risking damage or undesirable disassembly of the electronics pouch 416 or absorbent pouch 414. In an embodiment, the breakable connection piece contains an odor filter and a liquid-air separator. The breakable connection piece may also be manufactured from a porous polymer, e.g., a sintered polymer, which has been treated to provide liquid and odor blocking functions. For example, the breakable connection piece may include hydrophobic materials for liquid separation and activated carbon particles for odor control. In the case of a sintered polymer material, the breakable connection piece would not include an aperture, but would be a gas permeable structure having a sealed outer surface such that gas would be pulled through the breakable connection piece to transmit reduced-pressure. In such an embodiment, the outer surface of the breakable connection piece formed from the sintered polymer should be coated with a gas impermeable coating to provide a seal.

Where the intermediate cover member 450 is a breakable connection piece having a breakaway feature, the breakable connection piece may include an electrical connection. The electrical connection may electrically couple one or more sensors in the absorbent layer 410 to the processor of the electronics pouch 416. In such an embodiment, the reduced-pressure dressing 406 may include sensors to measure the fluid capacity of the dressing, the mechanical or pneumatic pressure at the tissue site, the pH of the wound, and other characteristics of the tissue site. The electrical coupling may also be used to provide power to a therapeutic system mounted within the absorbent layer 410 that requires power or monitoring, such as a wound camera or electrical stimulation system. In such an embodiment, a RF device, such as a RFID antenna, may be mounted in the reduced-pressure dressing 406 and the breakable connection piece may provide additional space to mount related electrical components. In addition, the breakable connection piece may provide multiple channels or lumens from the electronics pouch 416 to the absorbent pouch 414, which may enable the monitoring of pressure in specific areas of the reduced-pressure dressing. In such an embodiment, a feedback system may be used to determine absorbent saturation or other characteristics of a tissue site where substances are being delivered to a wound. In such an embodiment, the reduced-pressure dressing may be configured to deliver anti-microbial agents, analgesics, and cleansing solutions.

In another embodiment, the intermediate cover member 450 is an adhesive layer that provides a fluid seal between the electronics pouch 416 and absorbent pouch 414. The adhesive layer may be configured to allow the electronics pouch 416 to be separated from the absorbent pouch 414 by peeling the pouches apart.

Alternatively, the intermediate cover member 450 may be a film joined to the electronics pouch 416 and absorbent pouch 414 by a suitable method, such as bonding or welding. The film may be manufactured so that the film is weaker than the adjacent materials, thereby allowing the film to break instead of the adjacent electronics pouch 416 and absorbent pouch 414 as the pouches are pulled apart. Alternatively, a separation mechanism such as a string or strip of material is included beneath the film, such that pulling the string outward will cause the string or strip to unwind and tear the film to facilitate separation of the pouches.

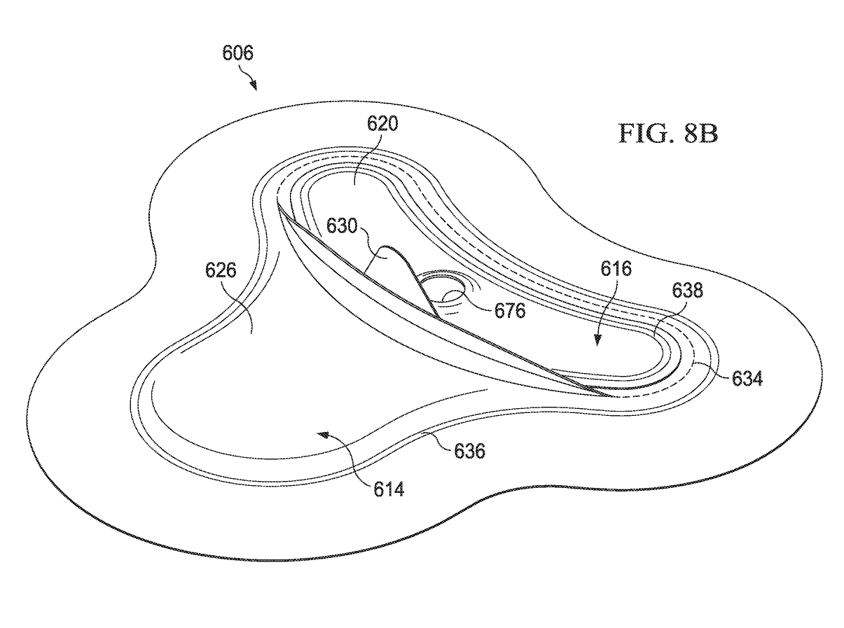

FIG. 7 shows another illustrative embodiment of a reduced-pressure dressing 506 that includes an electronics pouch 516 and an absorbent pouch 514. In the embodiment, reduced-pressure is transmitted from a pump 508 of the electronics pouch 516 to the absorbent pouch 514 via sealing member 554. The electronics pouch 516 is coupled to the absorbent pouch 514 by an intermediate cover member 550 that is manufactured from multiple parts, such as a first cover connector 562 and a second cover connector 564. The first cover connector 562 and second cover connector 564 are formed from different polymers so that adhesion between the first cover connector 562 and second cover connector 564 is strong enough to provide a fluid seal but weak enough to be easily broken. For example, if the first cover connector 562 is fabricated from polyurethane, then the second cover connector may be formed from polypropylene or high-impact polystyrene. In addition, the second cover connector 564 may be formed from polyurethane or another suitable material that is compatible with the pouch cover material and weldable using a HF welding process. In an embodiment, a fluid seal between the first cover connector 562 and second cover connector 564 is obtained by an interference fit between the connectors. As such, the first cover connector 562 and second cover connector 546 may be mating parts having a snap fit or twist-lock feature with sealing surfaces to maintain a fluid seal. The first cover connector 562 is coupled to the first cover 526 of the absorbent pouch 514 by an adhesive or weld, or by forming the first cover connector integrally to the first cover 526. The second cover connector 564 couples to the electronics pouch 516 in a similar manner.

In an embodiment having the first cover connector 562 and second cover connector 564, one of the parts, e.g., the first cover connector 562 may be manufactured by injection molding. The second cover connector 564 is then combined with the first cover connector 562 using an over-molding process. The over-molding process allows different materials to be used that are optimized for the joining process used at each interface. For example, the first cover connector 562 may be suitable for welding to a polymer surface of the absorbent pouch 514 while the second cover connector 564 is better suited for adhesive bonding to a surface of the electronics pouch 516 (e.g., to a polyimide or phenolic PCB substrate).

To form the second cover connector 564, the first cover connector 562 is installed in a mold, which is used to form the second cover connector 564 by over-molding the second cover connector 564 to the first cover connector 562. The over-molding process results in a part line 566 at the junction of the first cover connector 562 and second cover connector 564. The part line 566 may be formed such that when a separation force is applied to the pouches, the electronics pouch 516 separates from the absorbent pouch 514 along the part line 566. The part line 566 may be a flat surface or may include a mechanical interlock feature that enhances sealing. Where the fluid seal is enhanced by an interlock feature, the polymeric bond between the first cover connector 562 and second cover connector 564 is less important for the purposes of creating a fluid seal, and a weaker bond may be acceptable. As such, the first cover connector 562 and the second cover connector 564 may be formed from dissimilar materials that will not form a strong bond to one another. Also, a coating may be applied to the first cover connector 562 along the part line 566 to prevent the second cover connector 564 from permanently bonding to the first cover connector 562. In this way, the first cover connector 562 may be removably coupled to the second cover connector 564 to maintain a fluid seal until the electronics pouch 516 is separated from the absorbent pouch 514.

A fluid seal between the first cover connector 562 and second cover connector 564 may be more easily obtained by using an over-molding process than another manufacturing process because manufacturing tolerances and dimensional variations at the interface are negated by the over-molding process. The over-molding process also facilitates the joining of dissimilar materials. For example, in an embodiment in which there is a difference in hardness between the first cover connector 562 and the second cover connector 564, the connector formed from the softer polymer is formed using the over-molding process, while the opposing connector is formed using the injection molding process.

The use of dissimilar materials may also facilitate separation. Where the first cover connector 562 and second cover connector 564 include mechanical interlocking features, the softer connector may be more easily deformed to separate from the harder connector. In another embodiment both the first cover connector 562 and second cover connector 564 are injection molded and assembled together to provide a sealed coupling. In embodiments in which a more rigid part is manufactured from a material other than a thermoplastic (e.g., thermoset polymer), other manufacturing techniques may be employed.

In an embodiment, the absorbent pouch 514 remains in place at a tissue site while the electronics pouch 516 is removed to, for example, renew the power source of the pump 508. The power source of the pump 508 may be replaced within the electronics pouch 516 and the electronics pouch 516 may be reapplied or replaced with a new electronics pouch 516 to extend the life of the reduced-pressure dressing 506.

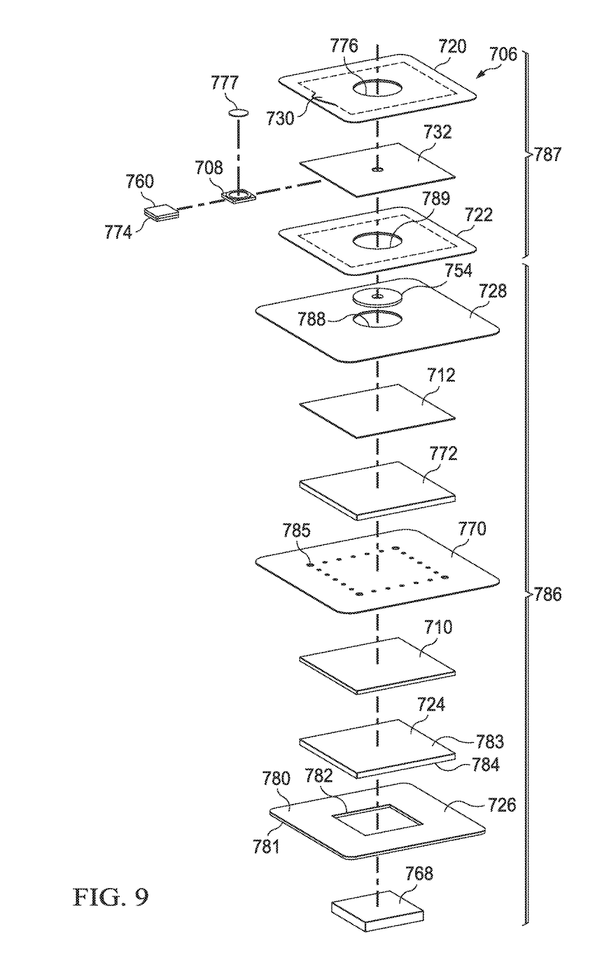

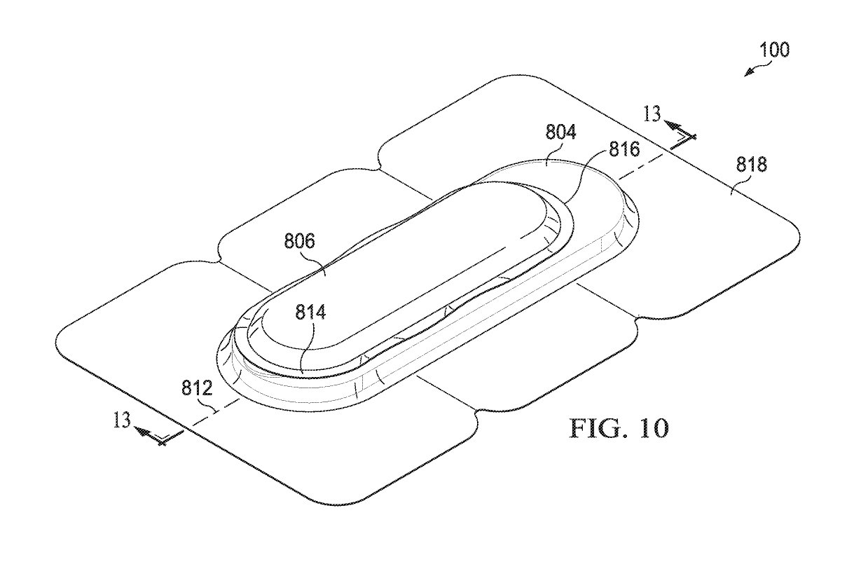

FIGS. 8A and 8B show an embodiment of a reduced-pressured dressing 606 having an arcuate shape. Aside from the arcuate shape, the reduced-pressure dressing 606 is generally analogous to the dressing of FIG. 1. For example, the reduced-pressure dressing 606 includes an absorbent pouch 614 having a first cover 626. The absorbent pouch 614 receives reduced-pressure from a pump that is housed within an electronics pouch 616. The electronics pouch 616 is removably coupled to absorbent pouch at a first bond 636 and a second bond 638. Adjacent the second bond 638, the reduced-pressure dressing 606 includes a perforation 634 that facilitates the separation of the absorbent pouch 614 from the electronics pouch 616. The first cover 620 of the electronics pouch 616 includes a tab 630 that can be pulled to initiate a tear along the perforation 634 to separate the pouches.

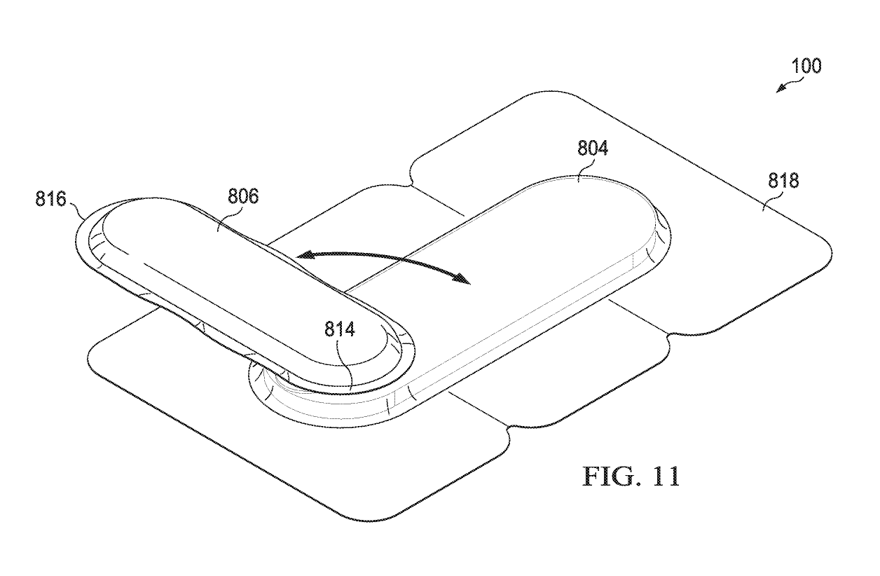

FIG. 9 shows an exploded view of a reduced-pressure dressing 706 that contains additional layers but is similar in many respects to the dressings discussed above. The reduced-pressure dressing 706 is shown in a rectangular form but may be formed to have any suitable shape for application to a tissue site. For example, the reduced-pressure dressing may be shaped to resemble the reduced-pressure dressing 606 of FIGS. 8A and 8B.

The reduced-pressure dressing 706 includes an optional intermediate manifold 768 that may be placed adjacent the tissue site, as discussed above. The reduced-pressure dressing 706 includes a first cover 726 and a second cover 728. The second cover 728 has a first side 780 and a second, patient-facing side 781. The second, patient-facing side 781 may be coated with a releasable adhesive to facilitate application to a tissue site. The second cover 728 also includes a treatment aperture 782 for placing over a portion of the tissue site (e.g., a wound) that receives reduced pressure. The reduced-pressure dressing 706 also includes a manifold layer 724, which is an internal distribution manifold having a first side 783 and a second, patient-facing side 784. In use, the manifold layer 724 distributes reduced-pressure to the tissue site. The second, patient-facing side 784 of the manifold layer 724 is coupled to the first side 780 of the second cover 728. An absorbent layer 710, which functions to receive and retain fluids from a tissue site, is coupled to the manifold layer 724.

A diverter layer 770 is coupled to the absorbent layer 710. The diverter layer 770 is disposed adjacent to the absorbent layer 710 and the manifold layer 724. The diverter layer 770 is formed from a liquid impermeable material but contains a plurality of apertures 785. The plurality of apertures 785 allow reduced pressure to be transmitted through the diverter layer 770 at desired locations. The diverter layer 770 helps control the pattern of reduced pressure as applied to the absorbent layer 710. The reduced pressure is distributed to the diverter layer 770 by a second manifold layer 772 that is coupled to the diverter layer 770. The apertures 785 may be arranged in a pattern for applying the reduced pressure to portions of the absorbent layer 710 to enhance the capability of the absorbent layer 710 to continue transferring reduced pressure to the tissue site as the absorbent layer 710 absorbs more fluid from the tissue site. The diverter layer 770 acts in conjunction with the second manifold layer 772 to ensure that the absorption capabilities and absorption efficiency of the absorbent layer 710 are increased relative to an absorbent layer 710 that is not used in conjunction with a diverter layer 770. By providing better distribution of liquid throughout the absorbent layer 710, the diverter layer 770 also increases the effective capacity and treatment time of the reduced-pressure dressing 706.

The diverter layer 770 may be made from any material that enhances the reduced pressure transmission and storage capabilities of an adjacent absorbent layer. For example, the diverter layer 770 may be made from a material that is substantially impermeable to liquid and gas and that diverts the reduced pressure to pass through apertures 785. Alternatively or in addition, the material from which the diverter layer 770 is made may have a predetermined moisture vapor transfer rate that is consistent with gas permeability. In either example, the diverter layer 770 may still include a pattern of apertures for transmitting a greater volume of liquid or gas than that permitted by a gas-permeable material not having apertures. It should be noted, however, that permeability of the diverter layer 770 to gas but not liquid may result in increased transmission of reduced pressure through the dressing while still directing liquid flow around or near the perimeter of the diverter layer 770.

In this embodiment, the reduced-pressure dressing 706 includes a liquid-air separator 712 coupled to the second manifold layer 772 and the first cover 726, which is coupled about the perimeter to the second cover 728. The first cover 726 includes an aperture 788 to receive reduced pressure. Together, the first cover 726 and second cover 728 form a first envelope 786 enclosing the manifold layer 724, absorbent layer 710, diverter layer 770, second manifold layer 772, and liquid-air separator 712.

To generate reduced pressure, the reduced-pressure dressing 706 includes a pump 708. The pump is mounted to a substrate 732 and coupled to a processor 760 and a power source 774. Additional electronic components may be coupled to the pump 708, processor 760, or power source 774 as desired. The substrate 732 is enclosed between a first electronics cover 720, which is coupled to a second electronics cover 722 to form a second envelope 787. The first electronics cover 720 also includes a vent 776 to fluidly couple an exhaust of the pump 708 to the external environment and an odor filter may be installed between the exhaust of the pump 708 and the vent 776 to prevent odor from a wound from escaping the reduced-pressure dressing 706. The substrate 732 and second electronics cover 722 also include apertures to facilitate the transmission of reduced pressure to the first envelope 786.

The second envelope 787 is removably coupled to the first envelope 786 using a removable coupling that provides a fluid seal. For example, a portion of the second electronics cover 722 may be coupled to a portion of the second cover 728. Optionally, a sealing member 754 provides a sealed fluid path between the second envelope 787 and the first envelope 786. The sealing member 754 includes an aperture for transmitting reduced-pressure generated by the pump 708 to the layers of the first envelope 786 for application to the tissue site.

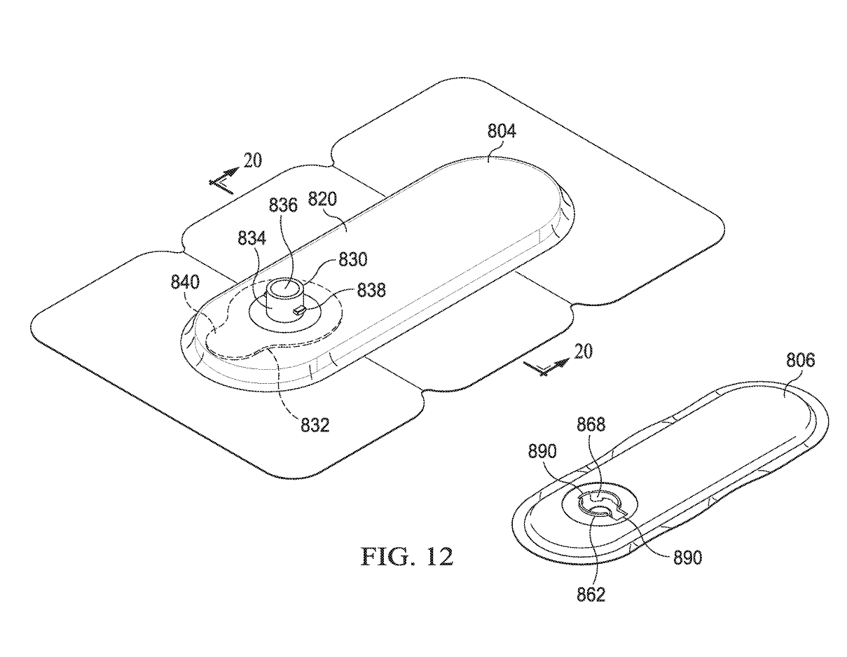

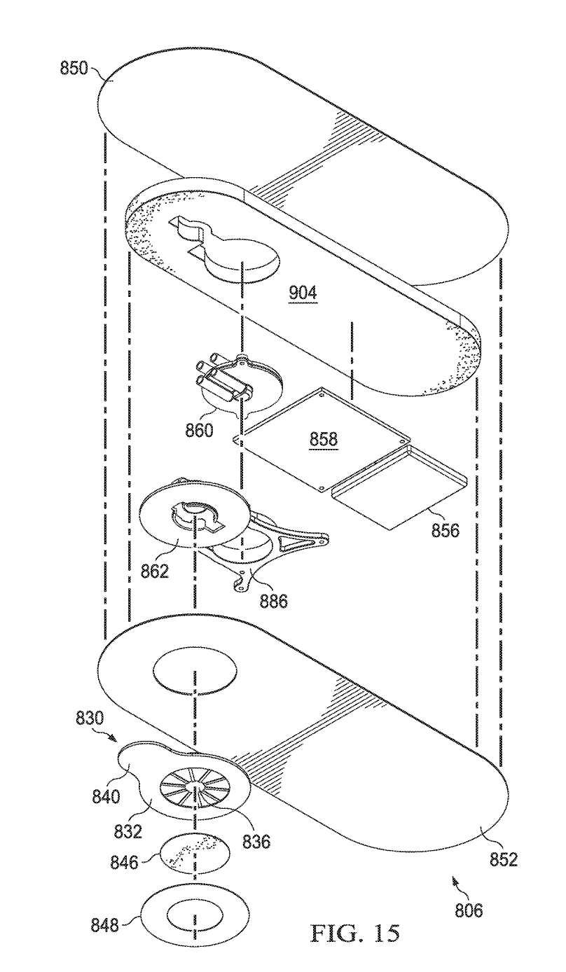

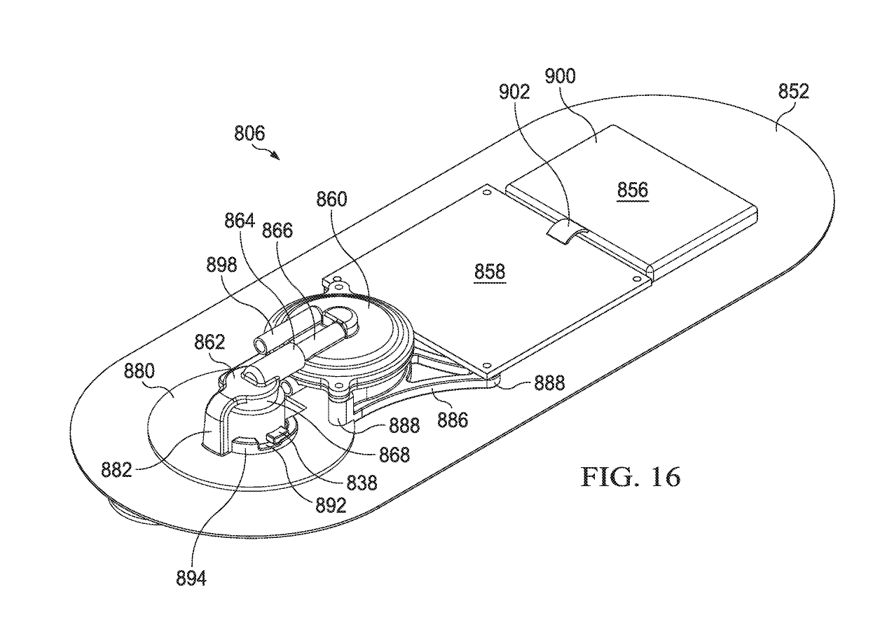

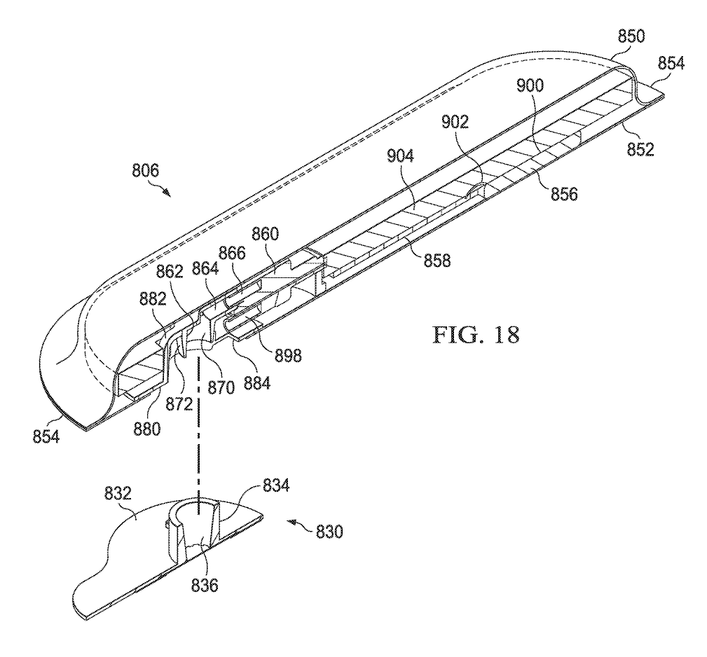

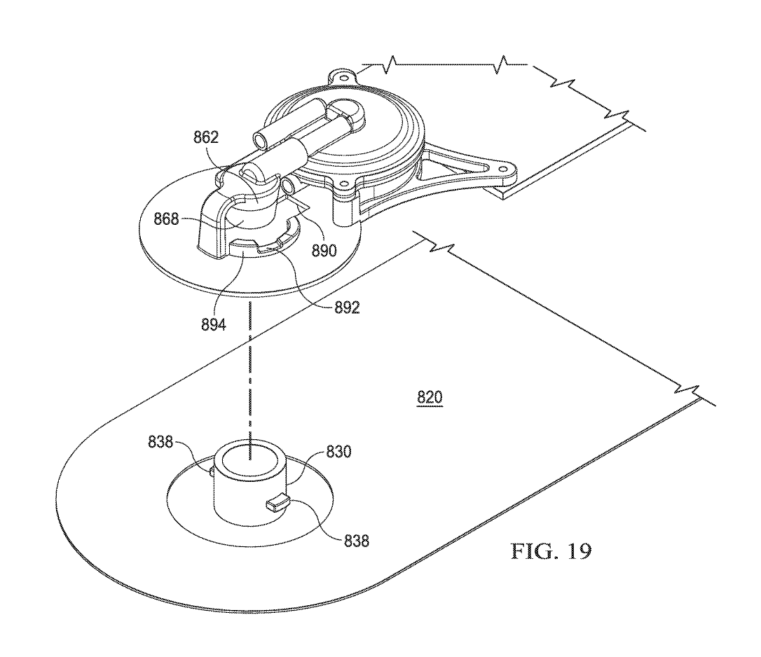

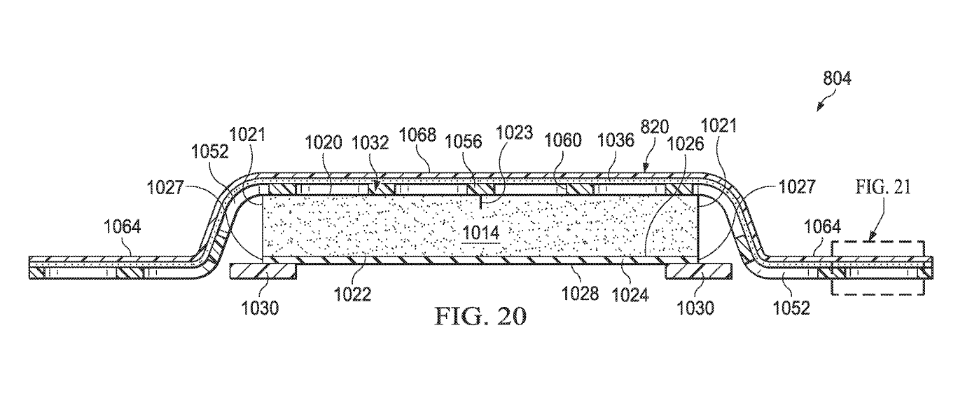

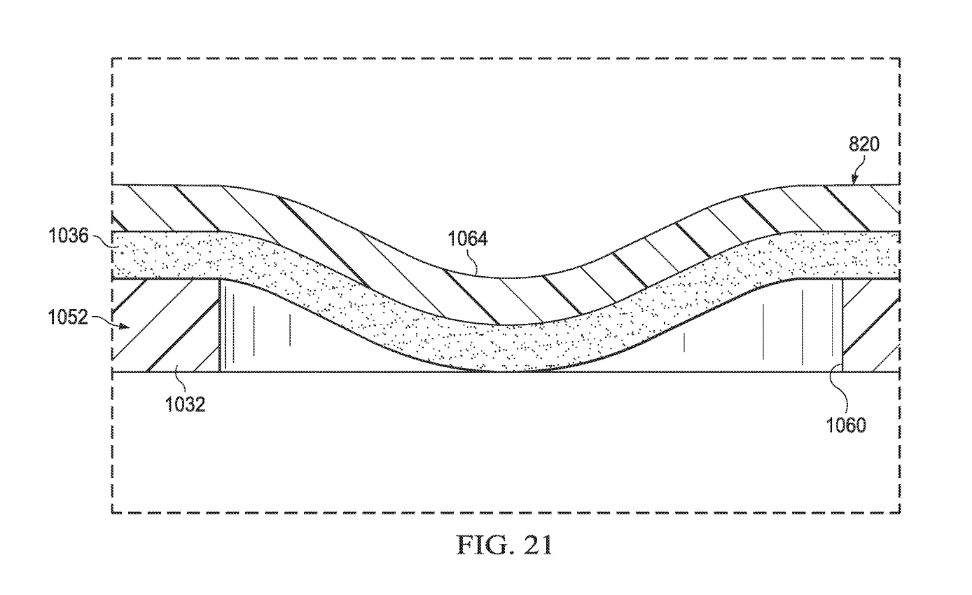

Another illustrative embodiment of the system 100 is shown in FIGS. 10-19. The system 100 may include a dressing 804 and a pump assembly 806, as illustrated in the example of FIG. 10. The dressing 804 may be in contact with tissue 808, covering a closed or sutured wound 810 (see FIG. 13) in some examples. The dressing 804 can also be applied to other types of tissue, such as the open wound of FIG. 1. The dressing 804 and pump assembly 806 are preferably longitudinally elongated along a common centralized axis 812 when fully attached to each other as shown in FIG. 10. Furthermore, a proximal end 814 and a distal end 816 of the dressing 804 and pump assembly 806 are arcuately rounded between generally straight and elongated lateral edges, however it should be appreciated that various polygonal or other shapes may be applied. In some examples, elongated dimensions of the dressing 804 and the pump assembly 806 may be at least twice a lateral width dimension, and the width dimension may be at least five times greater than thickness dimensions (measured away from the tissue).

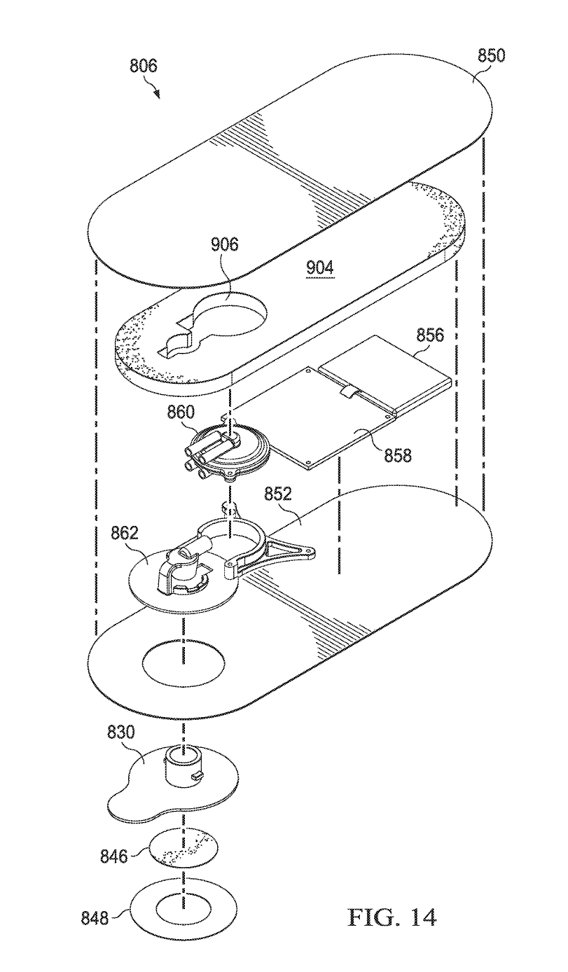

Referring to FIGS. 12 and 13, the dressing 804 may include an outer cover 820, an inner cover 822, a manifold 824, and at least one absorbent layer 826. A liquid-air separator 828 can also be provided between the absorbent layer 826 and the outer cover 820. The dressing 804 may optionally include a longitudinally and laterally enlarged drape or inner cover, such as an inner cover 818, for contacting a larger tissue area and achieving an improved seal.

In some embodiments, the outer cover 820 and the inner cover 822 may be made from a polymeric polyurethane film coated with an adhesive, such as an acrylic adhesive, by way of example and not limitation.

Furthermore, an exemplary material for the manifold 824 is a foam or non-woven material such as a compressed polyolefin from Essentra or a co-polyester from Libeltex, which can transport fluid from tissue into the absorbent layer 826. Additionally, mechanical properties or surface features of the manifold 824 may produce or transmit apposition forces to an incision to promote closure of the incision.

The absorbent layer 826 may comprise or consist essentially of a Texsus 500 gsm superabsorbent textile that can capture and store fluids. Alternately, an additional perforated film layer may be located between the absorbent layer 826 and the manifold 824, which can deter or prevent a backflow of liquid. A filter 846 can allow negative pressure transmission and block egress of liquids. In some examples, the filter 846 may be a hydrophobic filter, such as formed from Gore MMT 314 material that also acts as a viral and bacterial barrier.

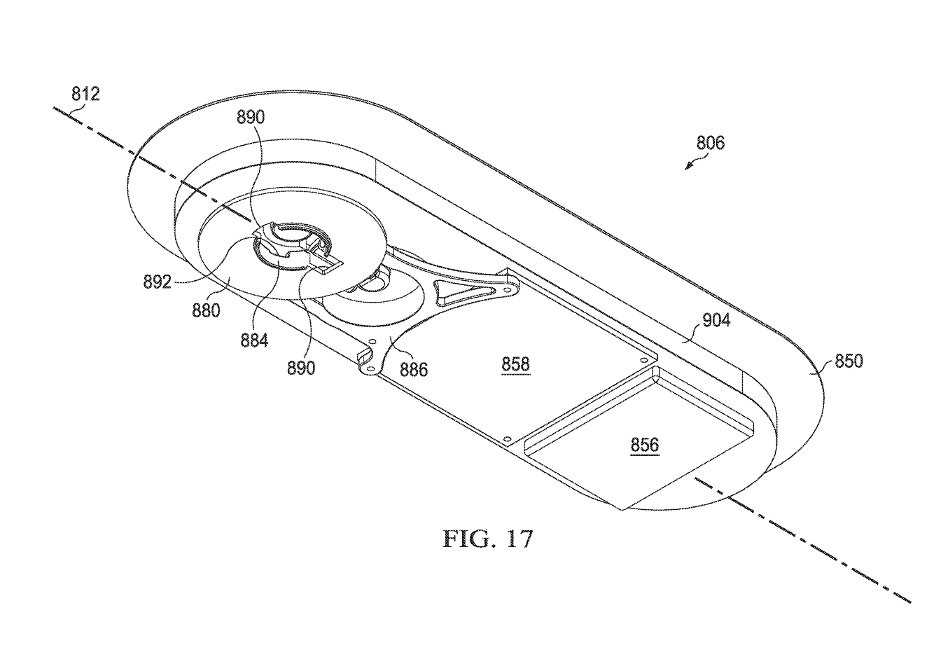

As illustrated in FIGS. 12-15, the dressing 804 may additionally include an outlet 830 having a flange 832 and body 834 centrally upstanding from the flange 832. The outlet 830 may be formed from a rigid polymer in some embodiments, and the body 834 may be generally cylindrical with an internal bore 836 having a slight taper. The bore 836 may be in fluid communication with the manifold 824, as illustrated in the example of FIG. 13. Two or more interlocking wings 838 may laterally project from opposite peripheral sides of the body 834. In the examples of FIGS. 12-15, a rounded and generally flat lip 840 may laterally project from a peripheral end of the flange 832. The filter 846 and a double-sided adhesive ring 848 can also be disposed within the dressing 804. The filter 846 and the ring 848 may be coaxially aligned with the bore 836 of the outlet 830, as illustrated in the example of FIG. 15.

Certain details of the pump assembly 806 can best be viewed with reference to FIGS. 12-19. The pump assembly 806 may include at least one outer cover 850 and an inner cover 852, which can be joined together at a peripheral edge 854. The outer cover 850 and the inner cover 852 are preferably made from a flexible polymeric material. The pump assembly 806 may further include a battery 856, a printed circuit board 858, and a pump 860.

An inlet 862 includes a conduit 864, which can be fluidly coupled to a rigid tube 866 mounted to an upper surface of the pump 860. An opposite end of the conduit 864 may be fluidly coupled to and adjacent to an inlet body 868. The body 868 may be a rigid body, having a bore 870 and an exterior surface 872. The bore 870 may be slightly tapered in some embodiments, and the exterior surface 872 may be tapered with a circular cross-section. The exterior surface 872 can fit within the matching taper of the bore 836 of the outlet 830 when coupled together.

The inlet 862 may also include a flange 880, which may be generally circular, flat and laterally extending. The flange 880 may be spaced from but rigidly affixed to the conduit 864 and the inlet 862 via an elongated arm 882. The elongated arm 882 upstands from one side of the flange 880 adjacent a central opening 884. A pump mount 886, which may include arcuately extending beams, may be integrally molded with the flange 880 on the inlet 862. The pump mount 886 may also provide bosses 888 for receiving screws or other fasteners for mounting the pump 860 and the printed circuit board 858 to the pump mount 886.

The inlet 862 may additionally include interlocking formations having receptacles 890 elongated along the longitudinal axis 812, creating keyhole portions accessible with the central hole 884, and also ledges 892 created by upper edges of partially circular walls 894 upstanding from the flange 880. The ledges 892 may be adjacent to and accessible by the receptacles 890. These interlocking formations can best be observed in FIGS. 12, 16, 17 and 19.

Alternately, each of the ledges 892 of the inlet 862 can be provided with a detent bump, groove, or barb to provide greater resistance for interlocking. A greater or lesser number of the wings 838, receptacles 890, and ledges 892 may be employed. Additionally or alternatively, rotational threads or linearly insertable snap fits and recesses can facilitate interlocking. The interlocking formations may be reversed in some examples, such that wings or the like may project from the inlet 862, and receptacles and ledges can be on the outlet 830.

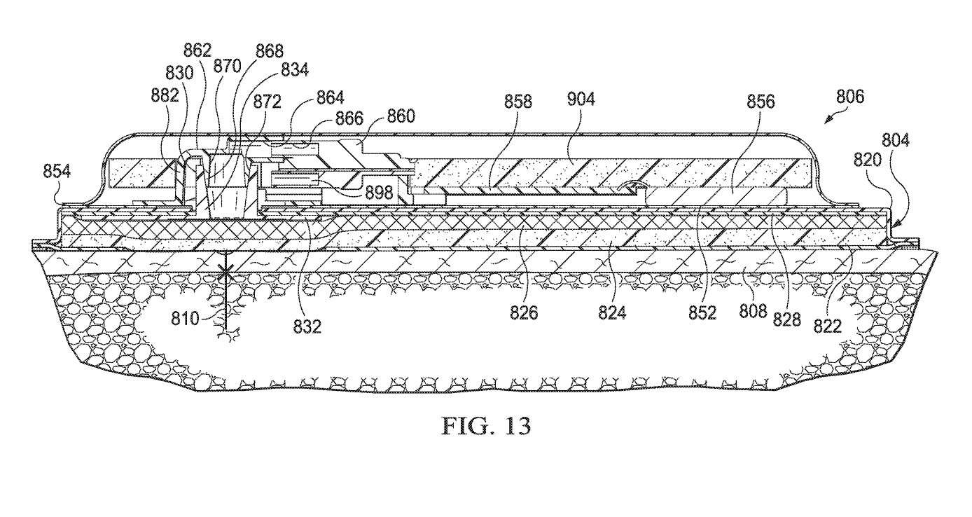

Engagement of the pump assembly 806 to the dressing 804 can be observed with reference to FIGS. 10, 11, 13 and 19. The connection is preferably made prior to placement of the dressing 804 onto a tissue site; however, the pump assembly 806 and the dressing 804 also may be connected after the dressing 804 is placed on tissue site. The inlet 862 of the pump assembly 806 may be coaxially aligned with the outlet 830 of the dressing 804, while a longitudinal direction of the pump assembly 806 is generally perpendicularly offset from the longitudinal axis 812 of the dressing 804. The wings 838 of the outlet 830 may be linearly inserted into the receptacles 890 of the inlet 862. The pump assembly 806 can be rotated approximately 90° toward the dressing 804 such that the pump assembly 806 and the dressing 804 are moved from the offset position of FIG. 11 to the aligned position shown in FIG. 10. The wings 838 can ride along and engage the ledges 892 during this rotational movement. If coupled together as illustrated in the examples of FIGS. 10 and 13, the inner cover 852 can directly contact a facing portion of the outer cover 820. Optionally, the ledges 892 may be provided with an increasingly angled and partly spiral taper to provide a camming action such that the body 834 and the body 868 are in sliding engagement and can provide a frictional interference seal, preferably without the need for an additional sealing element, although an optional O ring or the like may be provided.

The pump assembly 806 can be removed and decoupled from the dressing 804 by manual counter-rotation of the pump assembly 806 from the engaged position of FIG. 10 to the disengaged position of FIG. 11 and then linearly removed therefrom. In this exemplary construction, the pump assembly 806 can be discarded and replaced by a new pump assembly 806, the batteries 856 can be remotely recharged, or other maintenance may be conducted without removing the dressing 804 from a tissue site.

In some embodiments, the pump 860 may be a micro-pump, such as a piezoelectric pump, which can apply a voltage to an internal flexible disc to push air in one direction like a check valve. A micro-pump may be beneficial since it is extremely quiet to the user and others nearby. Venting tubes 898 may also extend from the pump 860.

FIGS. 13-18 illustrate the longitudinally offset arrangement of the printed circuit board 858 between the battery 856 and the pump 860. Furthermore, at least one laterally enlarged outer-facing surface, such as the surface 900, may be generally coplanar with the majority of the corresponding facing surface of the printed circuit board 858. Furthermore, a flexible electrical connection 902, such as a ribbon wire, can be provided between the battery 856 and the printed circuit board 858. The printed circuit board 858 may be rigidly affixed to the pump mount 886 in some embodiments, or the printed circuit board 858 may have a flexible electrical connection to the pump 860. The battery 856 and the printed circuit board 858 may have a generally rectangular periphery, as illustrated in the example of FIGS. 13-18. In some examples, the corners of the battery 856 and the printed circuit board 858 may be curved. The arrangement of the battery 856, the printed circuit board 858, and the pump 860 may provide a longitudinally flexible and thin configuration to better conform to a curved surface and also to allow movement of a joint such as a knee, hip, shoulder or elbow without inadvertent removal of the dressing 804. Alternately, the battery 856 can be stacked upon the printed circuit board 858, which may be above, below or to the side of the pump 860.

In some embodiments, a cushion 904 may be disposed between the outer cover 850 and the battery 856 and the printed circuit board 858. An internal aperture 906 within the cushion 904 may surround at least a majority of a periphery of the pump 860 and also the inlet 862. The cushion 904 may be longitudinally elongated and the aperture 906 may be offset adjacent a proximal end of the pump assembly 806. The cushion 904 is preferably flexible and breathable. For example, the cushion 904 may comprise or consist essentially of open-cell foam. The cushion 904 may protect internal components of the pump assembly 806 from external shocks and abuse during normal patient wear.

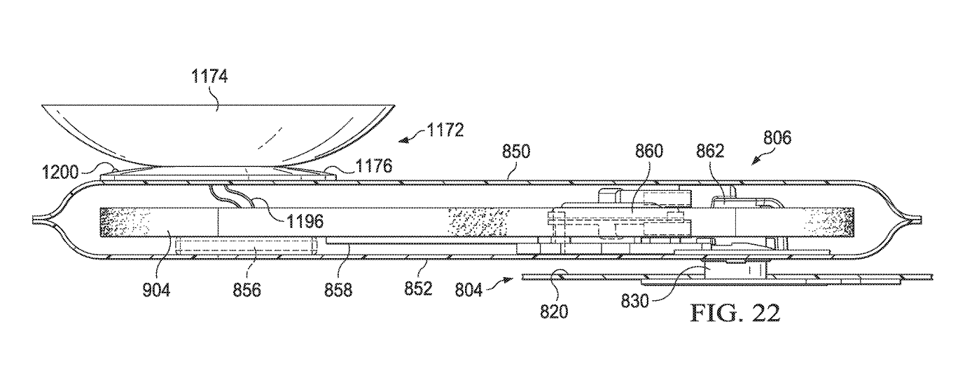

FIG. 20 is a schematic section view of another example embodiment of the dressing 804. In the example of FIG. 20, the dressing 804 may include a dressing bolster 1014. The dressing bolster 1014 may have a first side 1020, a periphery 1021, and a second side 1022. The second side 1022 of the dressing bolster 1014 may be configured to face a tissue site. The first side 1020 of the dressing bolster 1014 may be opposite the second side 1022 such that the first side 1020 may be configured to face outward or away from a tissue site. The periphery 1021 of the dressing bolster 1014 may define an outer boundary or lateral boundary of the dressing bolster 1014 and the first side 1020 and the second side 1022 of the dressing bolster 1014.

In some embodiments, the periphery 1021 of the dressing bolster 1014 may be an edge of the dressing bolster 1014, and may be a lateral edge positioned orthogonal relative to the second side 1022 of the dressing bolster 1014. The periphery 1021 of the dressing bolster 1014 may also be a beveled edge or an angled edge. The angled or beveled edge may help distribute shear stress, such as shear stress between the dressing bolster 1014 and epidermis.

In some embodiments, the dressing bolster 1014 may include one or more notches, recesses, or cuts, such as a notch 1023. For example, the notch 1023 may be a lateral or longitudinal cut in the dressing bolster 1014 on the first side 1020. The notch 1023 may enhance the flexibility of the dressing bolster 1014. Enhanced flexibility may be particularly useful for application of the dressing 804 over a joint or other area of movement. The notch 1023 may also take various shapes without limitation, such as, for example, hexagons, slits, or squares.

The dressing bolster 1014 may be formed from any suitable bolster material or manifold material. For example, the dressing bolster 1014 may be formed from a porous and permeable foam or foam-like material, a member formed with pathways, a graft, gauze, or any combination thereof. Negative pressure applied to the dressing bolster 1014 may enhance the permeability of the dressing bolster 1014.

In some embodiments, the dressing bolster 1014 may be an open-cell foam, such as reticulated polyurethane or polyether foam. Other suitable materials may include FXI technical foam (www.fxi.com), gauze, a flexible channel-containing member, a graft, and other similar materials. In some embodiments, ionic silver may be added to the material, such as, for example, by a micro bonding process. Other substances, such as antimicrobial agents, may also be added to the material.

In some embodiments, the dressing 804 may include a comfort layer 1024 having a first side 1026, a periphery 1027, and a second side 1028. The second side 1028 of the comfort layer 1024 may be configured to face a tissue site. The first side 1026 of the comfort layer 1024 may be opposite the second side 1028 such that the first side 1026 may be configured to face outward or away from a tissue site. The periphery 1027 of the comfort layer 1024 may define an outer boundary or lateral boundary of the comfort layer 1024. In some embodiments, the periphery 1027 of the comfort layer 1024 may be an edge of the comfort layer 1024.

The first side 1026 of the comfort layer 1024 may be coupled, for example, by a heat bond or other suitable technique to the second side 1022 of the dressing bolster 1014. In some embodiments, the periphery 1027 of the comfort layer 1024 may substantially correspond to, or be substantially aligned with, the periphery 1021 of the dressing bolster 1014. The comfort layer 1024 may enhance patient comfort when the dressing 804 is applied to epidermis of a patient. For example, in some embodiments, at least a portion of the second side 1028 of the comfort layer 1024 may be configured to directly contact the tissue site.

The comfort layer 1024 may be any material suitable for preventing skin irritation and discomfort while allowing fluid transmission through the comfort layer 1024. As non-limiting examples, a woven material, an elastic material, polyester knit textile substrate, a non-woven material, or a fenestrated film may be used. As another non-limiting example, an InterDry™ textile material from Milliken Chemical, a division of Milliken & Company, Inc. of Spartanburg, S.C., may be used. In some embodiments, the comfort layer 1024 may include antimicrobial substances, such as silver.

In some embodiments, the dressing 804 may include an interface seal 1030. In some embodiments, the interface seal 1030 may be a sealing ring. The interface seal 1030 may enhance or otherwise provide a fluid seal at or around dressing bolster 1014, the comfort layer 1024, or both. For example, the interface seal 1030 may help seal discontinuities, such as discontinuities between the outer cover 820 and epidermis at a tissue site. Further, the interface seal 1030 may also enhance the ability of the dressing 804 to impart an apposition force to a tissue site.

The interface seal 1030 may also function as a two-sided gasket that may provide a seal between the dressing 804 and epidermis. For example, the interface seal 1030 may provide a seal between the dressing bolster 1014, the comfort layer 1024, or the outer cover 820 and a tissue site. The interface seal 1030 may also absorb perspiration or other fluids from a tissue site. Further, the interface seal 1030 may distribute shear forces created, for example, by the application of negative pressure at the interface of the dressing bolster 1014 and epidermis.

The interface seal 1030 may be adapted to be positioned between the dressing bolster 1014 and a tissue site. For example, the interface seal 1030 may be positioned between the second side 1022 of the dressing bolster 1014 and a tissue site. In some embodiments, the interface seal 1030 may be coupled to the second side 1022 of the dressing bolster 1014.

In some embodiments, the interface seal 1030 may be positioned at the periphery 1021 of the dressing bolster 1014, or coupled to the periphery 1021 of the dressing bolster 1014. Further, the interface seal 1030 may be positioned between the dressing bolster 1014 and tissue at or around a tissue site, such as epidermis adjacent to a tissue site. In some embodiments, at least a portion of the interface seal 1030 may be positioned around the periphery 1021 of the dressing bolster 1014 and a periphery of a tissue site. Further, in some embodiments, at least a portion of the interface seal 1030 may substantially surround the periphery 1021 of the dressing bolster 1014 and a periphery of a tissue site.

The interface seal 1030 may be formed, as an illustrative example, by applying or bonding sealing material to the dressing bolster 1014. The sealing material that may be used for the interface seal 1030 may include hydrocolloids, hydrogels, silicone polymers (both crosslinked and un-crosslinked gels), and natural gums (xanthan, guar, cellulose). The sealing material may include other soft polymer gels, such as, for example, those based on polyurethanes, polyolefin gels, and acrylics.

The interface seal 1030 may have a material softness or hardness, between about 20 Shore OO to about 90 Shore OO. In some embodiments, the durometer of the interface seal 1030 may be between about 70 Shore OO to about 80 Shore OO. Further, the interface seal 1030 may have a modulus of elasticity that falls between a modulus of elasticity of the sealing member 1016 and a modulus of elasticity of a tissue site and/or epidermis.

The interface seal 1030 may have a width between about 10 millimeters to about 30 millimeters. In some embodiments, the width of the interface seal 1030 may be about 20 millimeters. The width of the interface seal 1030 may be directed, oriented, or adapted for positioning along a surface of a tissue site. In some embodiments, the width of the interface seal 1030 may extend beyond the periphery 1021 of the dressing bolster 1014 by about 10 millimeters and also overlap the second side 1022 of the dressing bolster 1014 by about 10 millimeters. Thus, the interface seal 1030 may straddle the periphery 1021 of the dressing bolster 1014, or otherwise extend beyond the periphery 1021 of the dressing bolster 1014. In other embodiments (not shown), the dressing bolster 1014 may entirely overlap the interface seal 1030.

The interface seal 1030 may have a thickness between about 0.3 millimeters to about 2.5 millimeters. In some embodiments, the thickness of the interface seal 1030 may be between about 0.7 millimeters to about 1.25 millimeters. The thickness of the interface seal 1030 may be perpendicular to the width of the interface seal 1030 and a tissue site.

The interface seal 1030 may be deployed by hand or extruded from an applicator, such as a syringe, prior to application of the dressing 804 to a tissue site. Sealing materials suitable for application by extrusion may include water soluble gums such as xanthan, guar, or cellulose, and thick greases, such as silicones. In other embodiments, the interface seal 1030 may be bonded in any suitable manner, such as, for example, by a heat bond, to the dressing 804 during manufacture. In some embodiments, the interface seal 1030 may have a ring-like or annular shape. In other embodiments, the interface seal 1030 may be linear. Further, in some embodiments, the interface seal 1030 may comprise one or more discrete members, including linear members, which may be formed into a ring-like or annular shape.

The interface seal 1030 may be coupled directly to another component of the dressing 804, or coupled with an attachment device, such as acrylic adhesive, cement, or other coupling device. For example, in some embodiments, the interface seal 1030 may be coupled to the second side 1022 of the dressing bolster 1014, and/or to an adjacent layer, such as the second side 1028 of the comfort layer 1024. Further, in some embodiments, the interface seal 1030 may be adapted to be positioned between the comfort layer 1024 and a tissue site, and/or tissue around a tissue site. In some embodiments, the comfort layer 1024 may be disposed between the dressing bolster 1014 and the interface seal 1030.

In some embodiments, the interface seal 1030 may include an absorbent. For example, the interface seal 1030 may be a hydrocolloid comprising an absorbent, such as carboxymethyl cellulose (CMC). The absorbent may permit the interface seal 1030 to absorb fluid from a tissue site in addition to enhancing the fluid seal around a tissue site. Including an absorbent in the interface seal 1030 may enhance the ability of the dressing 804 to manage and direct fluid away from a tissue site. For example, the dressing bolster 1014 may have a thickness between the first side 1020 and the second side 1022 of the dressing bolster 1014. The thickness of the dressing bolster 1014 may define at least a portion of a thickness of the dressing 804. The interface seal 1030 may be adapted to be positioned between the dressing 804 and tissue site, and around or surrounding a circumference, perimeter, or periphery of a tissue site.

Relative to other components of the dressing 804, the interface seal 1030 may be positioned, for example, around, on, or at the periphery 1021 of the dressing bolster 1014 and/or the comfort layer 1024. Further, the interface seal 1030 may be positioned around or surrounding a circumference of the dressing bolster 1014 and/or the comfort layer 1024. Further, the interface seal 1030 may be positioned around at least a portion of the dressing bolster 1014 or the comfort layer 1024 that is configured to be positioned directly against or in direct contact with a tissue site. At least a portion of the dressing bolster 1014 and/or the comfort layer 1024 may be exposed and configured to be positioned directly against a tissue site. Further, in such embodiments, the interface seal 1030 may surround the exposed portion of the dressing bolster 1014 and/or the comfort layer 1024.

The absorbent in the interface seal 1030 may wick or draw fluid in a lateral direction within the dressing 804, normal to the thickness of the dressing bolster 1014, and toward the periphery 1021 of the dressing bolster 1014 for absorption in the interface seal 1030. Fluid from a tissue site may be wicked or otherwise drawn in a lateral direction along the surface of a tissue site toward the periphery 1021 of the dressing bolster 1014 and into the interface seal 1030. Further, fluid from a tissue site may also flow through the dressing bolster 1014.

In some embodiments, the dressing 804 may include a base layer 1032, as illustrated in the example of FIG. 20. The use and configuration of the base layer 1032 in the dressing 804 may be beneficial for reducing the formation, size, and appearance of scars by, for example, increasing temperature and hydration levels at a tissue site. The base layer 1032 may also enhance the ability of the dressing 804 to impart apposition force to a tissue site, for example, for closing an incision or otherwise contracting a portion of a tissue site. The base layer 1032 may be configured to be coupled to the dressing bolster 1014 and/or tissue around a tissue site with an attachment device, such as an adhesive 1036. In some embodiments, a portion of the base layer 1032 may be configured to be coupled to the first side 1020 of the dressing bolster 1014.

The base layer 1032 may include a base layer flange 1052 configured to extend beyond the periphery 1021 of the dressing bolster 1014, for example, for coupling to tissue around or surrounding a tissue site. In some embodiments, the base layer flange 1052 may be configured to be positioned in direct contact with tissue around or surrounding a tissue site, such as epidermis. Further, the base layer flange 1052 may be positioned around or surrounding a central region 1056 of the base layer 1032. Thus, in some embodiments, the base layer flange 1052 may define, form, or be positioned at, a periphery of the base layer 1032. Further, the base layer flange 1052 may be configured to be positioned around the periphery 1021 of the dressing bolster 1014. In some embodiments, the base layer flange 1052 may be configured to substantially or entirely surround the periphery 1021 of the dressing bolster 1014.

The base layer 1032 may include a plurality of apertures 1060 disposed through the base layer 1032. In some embodiments, the apertures 1060 may be disposed through the central region 1056 of the base layer 1032, for example, to facilitate fluid communication with the dressing bolster 1014 and/or to couple the base layer 1032 to the dressing bolster 1014. In some embodiments, the apertures 1060 may be disposed through the base layer flange 1052, for example, to facilitate coupling the base layer 1032 to tissue around or surrounding a tissue site.

The central region 1056 of the base layer 1032 may be positioned adjacent to or proximate to the dressing bolster 1014, and the base layer flange 1052 may be positioned adjacent to or proximate to tissue surrounding a tissue site. The base layer flange 1052 may be positioned around or surrounding the dressing bolster 1014. Further, the apertures 1060 in the base layer 1032 may be in fluid communication with the dressing bolster 1014 and tissue around or surrounding a tissue site.

The apertures 1060 in the base layer 1032 may have any suitable shape, such as, for example, circles, squares, stars, ovals, polygons, slits, complex curves, rectilinear shapes, triangles, or other shapes. The apertures 1060 may be formed by cutting, by application of local RF energy, or other suitable techniques for forming an opening. Each of the apertures 1060 of the plurality of apertures 1060 may be substantially circular in shape, having a diameter and an area. The area of each of the apertures 1060 may refer to an open space or open area defining each of the apertures 1060. The diameter of each of the apertures 1060 may define the area of each of the apertures 1060. The area of the apertures 1060 described in the illustrative embodiments herein may be substantially similar to the area in other embodiments (not shown) for the apertures 1060 that may have non-circular shapes.

The diameter of each of the apertures 1060 may be substantially the same, or each of the diameters may vary depending, for example, on the position of the aperture 1060 in the base layer 1032. For example, the diameter of the apertures 1060 in the base layer flange 1052 may be larger than the diameter of the apertures 1060 in the central region 1056 of the base layer 1032. The diameter of each of the apertures 1060 may be between about 1 millimeter to about 50 millimeters. In some embodiments, the diameter of each of the apertures 1060 may be between about 1 millimeter to about 20 millimeters. The apertures 1060 may have a uniform pattern or may be randomly distributed on the base layer 1032. Further, in some embodiments, one or more of the apertures 1060 positioned adjacent to a corner may be smaller than the apertures 1060 positioned in the central region 1056. In some embodiments, the apertures adjacent to a corner may have a diameter between about 9.8 millimeters to about 10.2 millimeters, and the apertures 1060 in the central region 1056 may have a diameter between about 7.75 millimeters to about 8.75 millimeters.

The base layer 1032 may be a soft, pliable material. For example, the base layer 1032 may comprise a silicone gel, a soft silicone, hydrocolloid, hydrogel, polyurethane gel, polyolefin gel, hydrogenated styrenic copolymer gels, a foamed gel, a soft closed cell foam such as polyurethanes and polyolefins coated with an adhesive described below, polyurethane, polyolefin, or hydrogenated styrenic copolymers. The base layer 1032 may have a thickness between about 500 microns (μm) and about 1200 microns (μm). In some embodiments, the base layer 1032 may have a stiffness between about 5 Shore OO to about 80 Shore OO. The base layer 1032 may be comprised of hydrophobic or hydrophilic materials. The base layer 1032 may be operable to transmit forces, such as, for example, an apposition force, proximate to a tissue site, and to enhance a fluid seal with a tissue site.

In some embodiments (not shown), the base layer 1032 may be a hydrophobic-coated material. For example, the base layer 1032 may be formed by coating a spaced material, such as, for example, woven, nonwoven, molded, or extruded mesh with a hydrophobic material. The hydrophobic material for the coating may be a soft silicone, for example. In this manner, the spaced material may provide openings analogous to the apertures 1060.

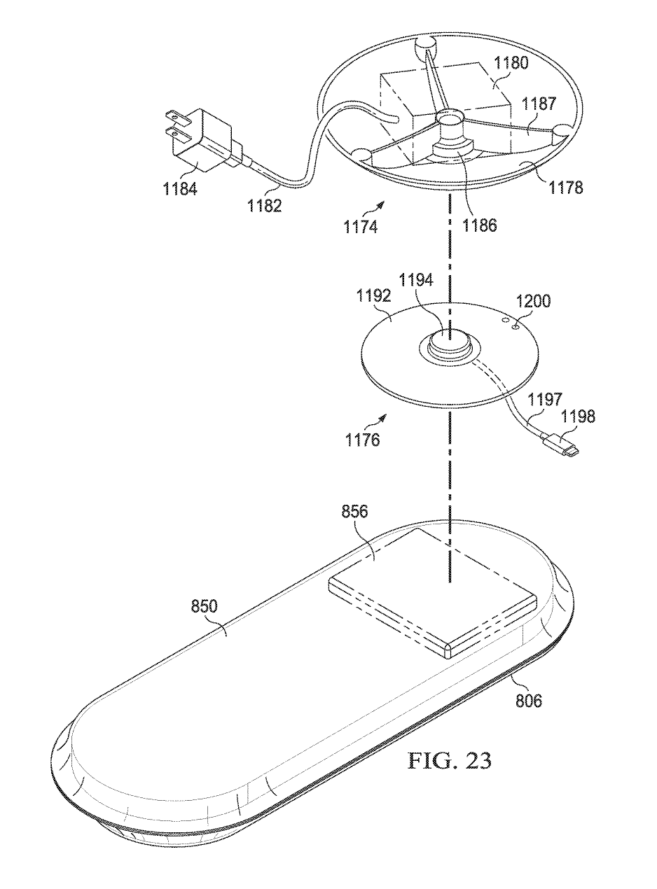

FIG. 21 is a detail view of the dressing 804 of FIG. 20, illustrating additional details that may be associated with some embodiments. For example, the adhesive 1036 may be in fluid communication with the apertures 1060 in at least the base layer flange 1052. In this manner, the adhesive 1036 may be in fluid communication with tissue surrounding a tissue site through the apertures 1060 in the base layer 1032. As illustrated in the example of FIG. 21, the adhesive 1036 may extend or be pressed through the plurality of apertures 1060 and contact epidermis, which can secure the dressing 804 to tissue surrounding a tissue site. The apertures 1060 may provide sufficient contact of the adhesive 1036 to epidermis to secure the dressing 804 about a tissue site. The configuration of the apertures 1060 and the adhesive 1036 may also permit release and repositioning of the dressing 804.

The size and configuration of the apertures 1060 may be designed to control the adherence of the dressing 804 to a tissue site. For example, the size and number of the apertures 1060 in corners of the base layer 1032 may be adjusted as necessary, depending on the chosen geometry of the corners, to maximize the exposed surface area of the adhesive 1036. Further, the apertures 1060 positioned near the corners may be fully housed within the base layer 1032, substantially precluding fluid communication in a lateral direction exterior to the corners. The apertures 1060 at the corners being fully housed within the base layer 1032 may substantially preclude fluid communication of the adhesive 1036 exterior to the corners, and may provide improved handling of the dressing 804 during deployment at a tissue site. Further, the exterior of the corners being substantially free of the adhesive 1036 may increase the flexibility of the corners to enhance comfort. The apertures 1060 may be adjusted in size and number to maximize the surface area of the adhesive 1036 in fluid communication through the apertures 1060 for a particular application or geometry of the base layer 1032.

The adhesive 1036 may be a medically-acceptable adhesive. The adhesive 1036 may also be flowable. For example, the adhesive 1036 may comprise an acrylic adhesive, rubber adhesive, high-tack silicone adhesive, polyurethane, or other adhesive substance. In some embodiments, the adhesive 1036 may be a pressure-sensitive adhesive comprising an acrylic adhesive with coating weight of 15 grams/m² (gsm) to 70 grams/m² (gsm). In some embodiments, the adhesive 1036 may be a layer having substantially the same shape as the base layer 1032. In some embodiments, the adhesive 1036 may be continuous layer. In other embodiments, the adhesive 1036 may be discontinuous. For example, the adhesive 1036 may be a patterned coating on a carrier layer, such as, for example, a side of the cover 820 adapted to face a tissue site. Further, discontinuities in the adhesive 1036 may be sized to control the amount of the adhesive 1036 extending through the apertures 1060 in the base layer 1032. The discontinuities in the adhesive 1036 may also be sized to enhance the moisture vapor transfer rate (MVTR) of the dressing 804.

Factors that may be utilized to control the adhesion strength of the dressing 804 may include the diameter and number of the apertures 1060 in the base layer 1032, the thickness of the base layer 1032, the thickness and amount of the adhesive 1036, and the tackiness of the adhesive 1036. An increase in the amount of the adhesive 1036 extending through the apertures 1060 may correspond to an increase in the adhesion strength of the dressing 804. A decrease in the thickness of the base layer 1032 may correspond to an increase in the amount of adhesive 1036 extending through the apertures 1060. Thus, the diameter and configuration of the apertures 1060, the thickness of the base layer 1032, and the amount and tackiness of the adhesive 1036 utilized may be varied to provide a desired adhesion strength for the dressing 804. In some embodiments, the thickness of the base layer 1032 may be about 200 microns, the adhesive 1036 may have a thickness of about 30 microns and a tackiness of 2000 grams per 25 centimeter wide strip, and the diameter of the apertures 1060 in the central region 1056 of the base layer 1032 may be about 10 millimeters. In some embodiments, the tackiness of the adhesive 1036 may vary in different locations of the base layer 1032. For example, in locations of the base layer 1032 where the apertures 1060 may be comparatively larger the adhesive 1036 may have a lower tackiness than other locations of the base layer 1032.

The cover 820 may have a periphery 1064 and a central region 1068. The periphery 1064 may be positioned proximate to the base layer flange 1052 in some examples. As illustrated in the example of FIG. 20, the adhesive 1036 may be positioned at least between the periphery 1064 of the cover 820 and the base layer flange 1052. In some embodiments, a portion of the periphery 1064 may extend beyond the base layer flange 1052 and into direct contact with tissue surrounding a tissue site. The adhesive 1036 may also be positioned at least between the periphery 1064 of the 820 and tissue, such as epidermis surrounding a tissue site. In some embodiments, the adhesive 1036 may be disposed on a surface of the cover 820 adapted to face the base layer 1032.

In some embodiments, the cover 820 may be configured to extend beyond the periphery 1021 of the dressing bolster 1014. Further, in some embodiments, the cover 820 may be configured to cover at least a portion of the first side 1020 of the dressing bolster 1014 and to extend beyond the periphery 1021 of the dressing bolster 1014 proximate to the base layer flange 1052. In some embodiments, the adhesive 1036 may be positioned between the cover 820 and the base layer 1032 such that the adhesive 1036 is in fluid communication with at least the apertures 1060 in the base layer flange 1052. The adhesive 1036 may be positioned at least between the cover 820 and the base layer flange 1052. Further, in some embodiments, the adhesive 1036 may be configured to be in fluid communication with tissue around or surrounding a tissue site through the apertures 1060 in the base layer flange 1052.

The outer cover 820 in the example embodiment of FIG. 20 may be formed from any suitable material that allows for a fluid seal. A fluid seal may be a seal adequate to maintain reduced pressure at a desired site given the particular reduced pressure source or system involved. The outer cover 820 may comprise, for example, one or more of the following materials: hydrophilic polyurethane; cellulosics; hydrophilic polyamides; polyvinyl alcohol; polyvinyl pyrrolidone; hydrophilic acrylics; hydrophilic silicone elastomers; an INSPIRE 2301 material from Expopack Advanced Coatings of Wrexham, United Kingdom having, for example, an MVTR (inverted cup technique) of 14400 g/m²/24 hours and a thickness of about 30 microns; a thin, uncoated polymer drape; natural rubbers; polyisoprene; styrene butadiene rubber; chloroprene rubber; polybutadiene; nitrile rubber; butyl rubber; ethylene propylene rubber; ethylene propylene diene monomer; chlorosulfonated polyethylene; polysulfide rubber; polyurethane (PU); EVA film; co-polyester; silicones; a silicone drape; a 3M Tegaderm® drape; a polyurethane (PU) drape such as one available from Avery Dennison Corporation of Pasadena, Calif.; polyether block polyamide copolymer (PEBAX), for example, from Arkema, France; Expopack 2327; or other appropriate material.

The outer cover 820 may be vapor permeable and liquid impermeable, thereby allowing vapor egress and inhibiting liquid egress. In some embodiments, the outer cover 820 may be a flexible, breathable film, membrane, or sheet having a high moisture vapor transfer rate of, for example, at least about 300 g/m² per 24 hours. In other embodiments, a low or no vapor transfer drape might be used. The outer cover 820 may comprise a range of medically suitable films having a thickness between about 15 microns (μm) to about 50 microns (μm).

In some embodiments, a portion of the base layer 1032 may be configured to be positioned on or coupled to the second side 1022 of the dressing bolster 1014. For example, the central region 1056 of the base layer 1032 may be positioned on or coupled to the second side 1022 of the dressing bolster 1014. In some embodiments, the base layer 1032 may be configured to be positioned between the dressing bolster 1014 and a tissue site. Further, in some embodiments, the base layer 1032 may be positioned or coupled relative to the dressing bolster 1014 through other elements, such as, for example, the comfort layer 1024 and/or the interface seal 1030. For example, the comfort layer 1024 may be positioned between the second side 1022 of the dressing bolster 1014 and the base layer 1032. In other embodiments, the base layer 1032 may be directly positioned on or directly coupled to the dressing bolster 1014.

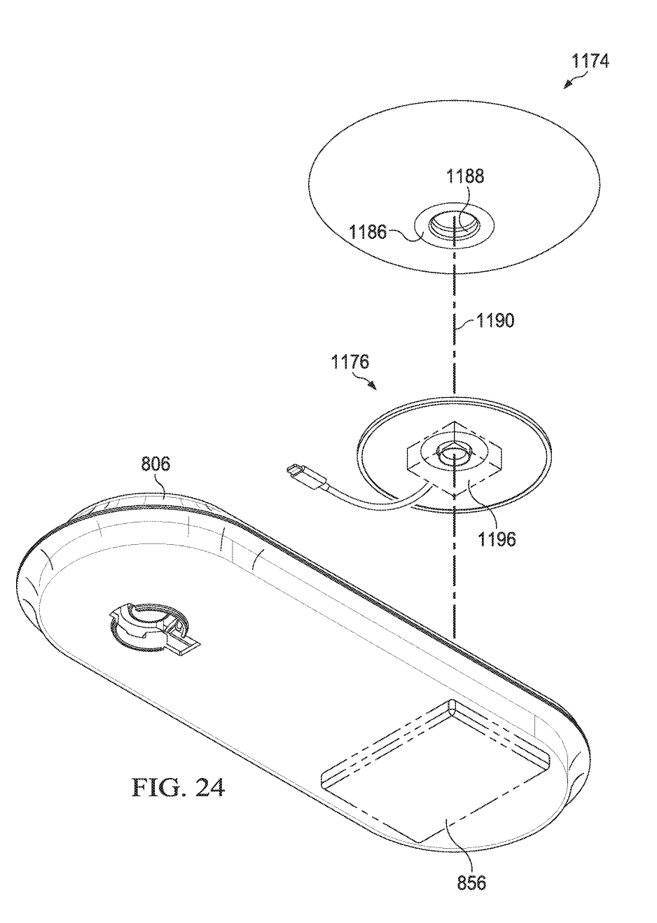

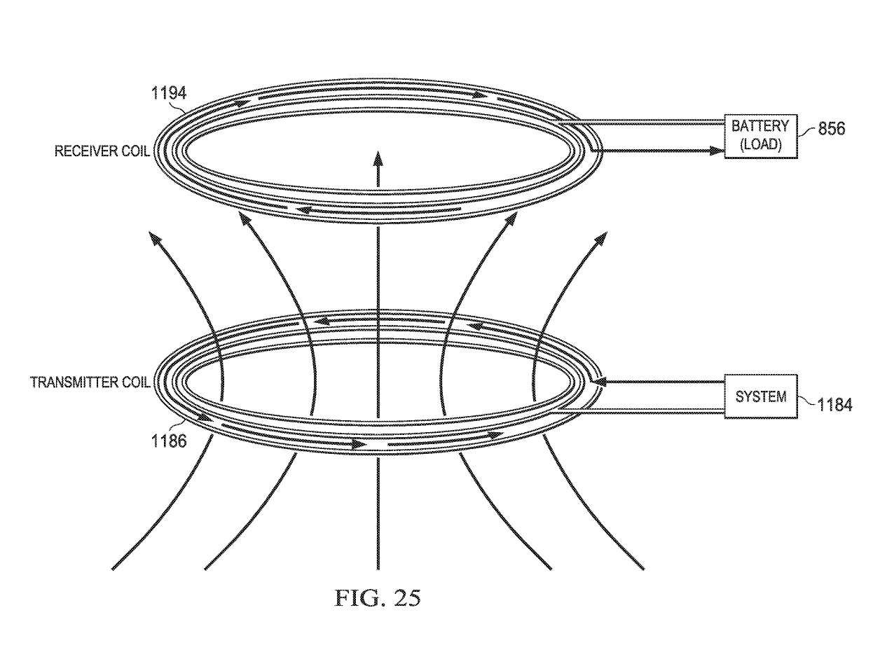

Another example embodiment of the pump assembly 806 is illustrated in FIGS. 20-23. In the example of FIG. 20, the pump assembly may include an electrical charging assembly 1172, which may include a base charger 1174 and a receiver 1176. As illustrated in FIG. 21, the base charger 1174 may include a partially spherical support base 1178, a transmitter electronics module 1180, an electrical wire 1182, a wall outlet plug 1184 and an antenna 1186. The base 1178 may also include reinforcement ribs 1187 radiating outwardly from a center of the base 1178. The base charger 1174 is preferably manufactured from an injection molded polymeric material. Furthermore, antenna 1186 is preferably a permanent magnet formed in an annular ring with a central opening 1188 coaxially aligned with a central axis 1190 of the base 1178, as illustrated in the example of FIG. 22.

In some embodiments, the receiver 1176 may include an injection-molded polymeric outer surface 1192 which may be frustoconical or partially spherical. An antenna 1194 may be mounted within the receiver 1176 and electrically connected to a receiver electronics module 1196. As illustrated in FIG. 21 and FIG. 22, the antenna 1194 may be centrally mounted, and the receiver electronics module 1196 may be disposed on an opposite side of the receiver 1176. The antenna 1194 is preferably a permanent magnet, such as a ring magnet with a central hole aligned with the axis 1190. The wire 1197 and the connector 1198 electrically connect the receiver electronics module 1196 to the battery 856 through the outer cover 850.

The antenna 1186 and the antenna 1194 can advantageously serve a multifunctional purpose, including removeably securing together a transmitter base and receiver during charging and also inductively transmitting an electrical current charge between the base transmitter and the receiver when energized. In some embodiments, 15 watts or less of power may be inductively transferred between the antenna 1186 and the antenna 1194, the antenna 1186 and the antenna 1194 may be configured to have an air gap of three millimeter or less. The receiver 1176 can be adhesively or otherwise permanently affixed to the outer cover 850 of the pump assembly 806 in some embodiments. In some examples, all or part of the receiver 1176 may be enclosed with the outer cover 850.

One or more LED lights 1200 or an audible sounder is optionally mounted on the receiver 1176 or the flexible outer cover 850, and electrically connected to the printed circuit board 858. This external feedback interface can provide an operator with an indication of the battery power status, a fluid leakage alert, a pump operation alert or the like. Additionally or alternatively, feedback may be provided by a haptic device employing a motor, drive and out-of-balance cam, which can selectively create a vibration that can be felt by an operator but not apparent to others in the vicinity.

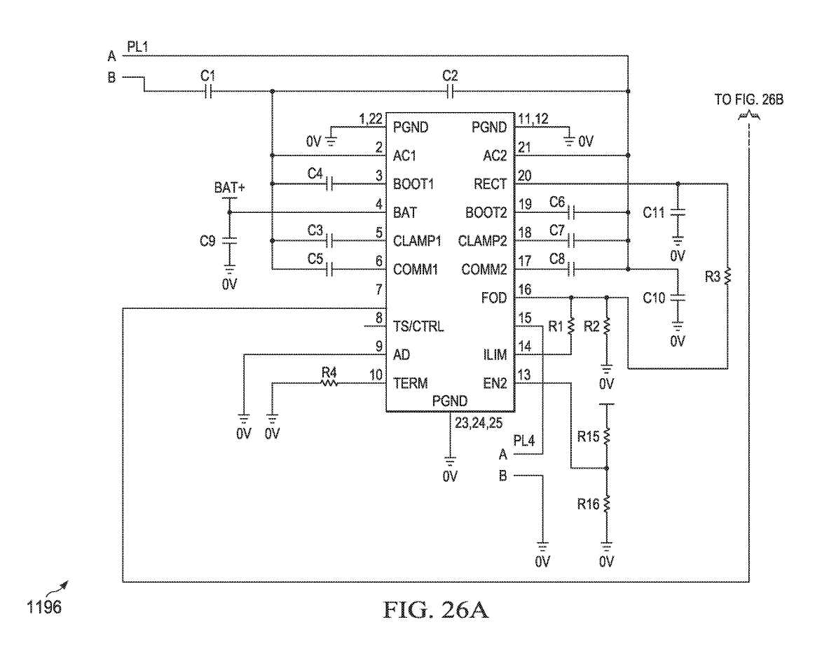

FIGS. 24A and B illustrate an exemplary electric circuit diagram for the inductive charging configuration. The electrical circuit for the wireless power transmitter or the base charger 1174 is preferably constructed with the major electronic components identified in the following Table 1, which are mounted to conductive traces.

TABLE 1

| No. | Reference No. | Component Name | Quantity |
|---|---|---|---|
| 1. | H7184 | ROHS COMPLIANT BARE PCB | 1 |
| 2. | H7188 | TRANSMITTER COIL | 1 |
| 3. | H7186 | PIC PROGRAMMED | 1 |
| 4. | U2 | FT232 USB TO UART IC | 1 |
| 5. | U3 | BQ50002 WIRELESS POWER AFE IC | 1 |
| 6. | | | |
| 7. | U5 | BQ500511 WIRELESS POWER CONT | 1 |
| 8. | U6 | CR95HF NFC TRANSCEIVER IC | 1 |
| 9. | | | |
| 10. | | | |
| 11. | | | |
| 12. | | | |
| 13. | | | |
| 14. | R1, R23-25, R33 | 4.7k 1% 0603 RESISTOR | 5 |
| 15. | R2-7, R32, R36, R49 | 180 R 1% 0603 RESISTOR | 9 |
| 16. | R9, R13, R17, R40, R41, | 10k 1% 0603 RESISTOR | 14 |
| | R42, R43, R44, R45, R48, | | |
| | R52, R53, R55, R56 | | |
| 17. | R10 | 20mR 1% 0603 RESISTOR | 1 |
| 18. | R11 | 76K8 1% 0603 RESISTOR | 1 |
| 19. | | | |
| 20. | R12 | 2R 1% 0603 RESISTOR | 1 |
| 21. | R14, R15 | 200mR 1% 0603 RESISTOR | 2 |
| 22. | R16 | 69.8k 1% 0603 RESISTOR | 1 |
| 23. | R26, R27, R28, R41, R50 | 100k 1% 0603 RESISTOR | 5 |
| 24. | R29 | 113k 1% 0603 RESISTOR | 1 |
| 25. | | | |
| 26. | R30 | 80.6k 1% 0603 RESISTOR | 1 |
| 27. | R35 | 24.9k 1% 0603 RESISTOR | 1 |
| 28. | R34 | 7.5k 1% 0603 RESISTOR | 1 |
| 29. | R37, R38 | 330R 1% 0603 RESISTOR | 2 |
| 30. | R46 | 10K NTC THERMISTOR | 1 |
| 31. | | | |
| 32. | R47 | 499k 1% 0603 RESISTOR | 1 |
| 33. | R8, R51 | 1k 1% 0603 RESISTOR | 2 |
| 34. | R54 | 3.3k 1% 0603 RESISTOR | 1 |
| 35. | | | |
| 36. | | | |
| 37. | | | |
| 37. | | | |
| 38. | | | |
| 39. | | | |
| 40. | C1, C5, C11, C13-15, C17, | 100 nF, 50 V, X7R 0603 CERAM, CAPACITOR | 20 |
| | C19-21, C25, C27-28, C31, | | |
| | C36-37, C40, C44, C50-51 | | |
| 41. | C2 | 2.2 nF, 200 V, X7R 0603 CERAM, CAPACITOR | 1 |
| 42. | C6-7 | 2.2 uF, 10 V, X7R 0603 CERAM, CAPACITOR | 2 |
| 43. | C8 | 22 nF, 100 V, X7R 0603 CERAM, CAPACITOR | 1 |
| 44. | C9, C24 | 1 uF, 16 V, X7R 0603 CERAM, CAPACITOR | 2 |
| 45. | | | |
| 46. | C10, C12 | 22 uF, 10 V, X5R 0603 CERAM, CAPACITOR | 2 |
| 47. | C18, C26 | 47 nF, 100 V, X7R 0603 CERAM, CAPACITOR | 2 |
| 48. | C22 | 2.7 nF, 50 V, X7R 0603 CERAM, CAPACITOR | 1 |
| 49. | C23, C49 | 1 nF, 100 V, X7R 0603 CERAM, CAPACITOR | 2 |
| 50. | C29 | 470 nF, 16 V, X7R 0603 CERAM, CAPACITOR | 1 |
| 51. | | | |
| 52. | C30, C33 | 10 pF, 50 V, C0G 0603 CERAM, CAPACITOR | 2 |
| 53. | C32, C39 | 180 pF, 50 V, X7R 0603 CERAM, CAPACITOR | 2 |
| 54. | C34, C39 | 10 nF, 50, X7R 0603 CERAM CAPACITOR | 2 |
| 55. | C34, C45 | 4.7 uF, 25 V, CASE A TANTALUM CAPACITOR | 2 |
| 56. | C41, C48 | 150 pF, 200 V, C0G 0603 CERAM, CAPACITOR | 2 |
| 57. | | | |
| 58. | | | |
| 59. | C42, C47 | 82 pF, 200 V, C0G 0603 CERAM CAPACITOR | 2 |
| 60. | C43 | 2.2 uF, 10 V, 1206(3216) A TANT CAPACITOR | 1 |
| 61. | C46 | 1 uF, 6.3 V EMI FILTER CAPACITOR | 1 |
| 62. | C52 | 100 uF, 6.3 V, 1206(3216) A TANT CAPACITOR | 1 |
| 63. | | | |
| 64. | | | |
| 65. | | | |
| 66. | | | |
| 67. | L1, L3-4 | 0.5R, 0.2 A, 0805 FERRITE BEAD | 3 |
| 68. | L2 | 0.6R, 0.2 A, 0805 FERRITE BEAD | 1 |
| 69. | | | |
| 70. | | | |
| 71. | Q1 | P CHANNEL, TO-236 MOSFET | 1 |
| 72. | | | |
| 73. | | | |
| 74. | D1-2 | 30 V, 200 mA, SOD-523 DIODE | 2 |
| 75. | | | |
| 76. | | | |
| 77. | LED1-4, LED7-8 | GREEN LED | 6 |
| 78. | LED5, LED9 | YELLOW LED | 2 |
| 79. | LED6 | ORANGE LED | 1 |
| 80. | | | |
| 81. | | | |
| 82. | XTAL1 | 27.12 MHz OSCILLATOR | 1 |
| 83. | | | |
| 84. | | | |
| 85. | F1 | 2 A, 0603 FUSE | 1 |
| 86. | | | |
| 87. | | | |
| 88. | BUZ1 | PIEZO BUZZER | 1 |
| 89. | | | |
| 90. | | | |
| 91. | J15 | MICRO USB CONNECTOR | 1 |
| 92. | | | |
| 93. | | | |
| 94. | PL15 | POWER TRANSMITTER COIL | 1 |
| 95. | | | |
| 96. | J25 | DC POWER SOCKET | 1 |
| 97. | | | |
| 98. | R39, 41-44, 52-53, 55-56 | NO FIT | |
| 99. | C24 | | |
| 100. | J3, PL2, PL4, U4 | | |

Electronics of the electrical circuit within the receiver electronics module 1196 are preferably as set forth in the following Table 2.

TABLE 2

| No. | Reference No. | Component Name | Quantity |
|---|---|---|---|
| 1. | H7183 | ROHS COMPLIANT BARE PCB | 1 |
| 2. | H7187 | RECEIVER COIL | 1 |
| 3. | H7185 | PIC PROGRAMMED | |
| 4. | | | |
| 5. | U1 | M24SR04 NFC TRANSPONDER | 1 |
| 6. | U2 | BQ51050 WIRELESS POWER RECEIVER | 1 |
| 7. | | | |
| 8. | | | |
| 9. | R1 | 330R 1% 0603 RESISTOR | 1 |
| 10. | R2 | 220R 1% 0603 RESISTOR | 1 |
| 11. | R4 | 2.2k 1% 0603 RESISTOR | 1 |
| 12. | R5, R6, R15 | 10k 1% 0603 RESISTOR | 3 |
| 13. | R7-11 | 180R 1% 0603 RESISTOR | 5 |
| 14. | R19-23, R27 | 100k 1% 0603 RESISTOR | 6 |
| 15. | R25 | 1.2k 1% 0603 RESISTOR | 1 |
| 16. | R26 | 1k 1% 0603 RESISTOR | 1 |
| 17. | | | |
| 18. | C1 | 150 nF, 25 V, 0603 CERAM, CAPACITOR | 1 |
| 19. | C2 | 3.3 nF, 50 V, 0603 CERAM, CAPACITOR | 1 |
| 20. | C3, C7 | 470 nF, 16 V, 0603 CERAM, CAPACITOR | 2 |
| 21. | C4, C6 | 10 nF, 50 V, 0603 CERAM, CAPACITOR | 2 |
| 22. | C5, C8 | 47 nF, 100 V, 0603 CERAM, CAPACITOR | 2 |
| 23. | C9, C12, C13 | 100 nF, 50 V, 0603 CERAM, CAPACITOR | 3 |
| 24. | C11 | 10 uF, 6.3 V, 0603 CERAM, CAPACITOR | 1 |
| 25. | | | |
| 26. | Q1 | P CHANNEL MOSFET | 1 |
| 27. | | | |
| 28. | F1 | 2 A 0603 FUSE | 1 |
| 29. | | | |
| 30. | LED2-5 | GREEN LED | 4 |
| 31. | LED6 | ORANGE LED | 1 |
| 32. | | | 1 |
| 33. | PL1 | POWER RECEIVER COIL | 1 |
| 34. | | | |
| 35. | R3, R14, R16, C10, PL3-6 | | |

In some examples, the receiver electronic components may be alternately mounted directly onto the printed circuit board 858 or on a separate printed circuit board also within the pump cover. The electronic circuits within the transmitter electronics module 1180, receiver electronics module 1196 and printed circuit board 858 are all preferably of a printed circuit board construction with one or more microprocessors and memory. In other examples, any or all of the circuits may be alternately constructed in a hard wired or stamped metal conductor arrangement.

A wireless communications transmitter and/or receiver can be included in the pump's electrical circuit in some embodiments, which can connect the pump controller to a remote computer or smart phone for pump monitoring, therapy logging, pump information notifications or alerts, and/or changes to the pump operation. Additional sensors (such as accelerometers, temperature sensors, flow sensors, etc.) can also be included to sense charging, electrical operation, and pumping performance, which can be electrically connected to the PCB microcontroller and/or circuit. Optionally, an on-off switch may be actuated or deactivated through mechanical engagement or disengagement of the pump assembly and dressing interlocking formations or of other physical contact of surfaces thereof; thus, the switch may be a limit or proximity switch.

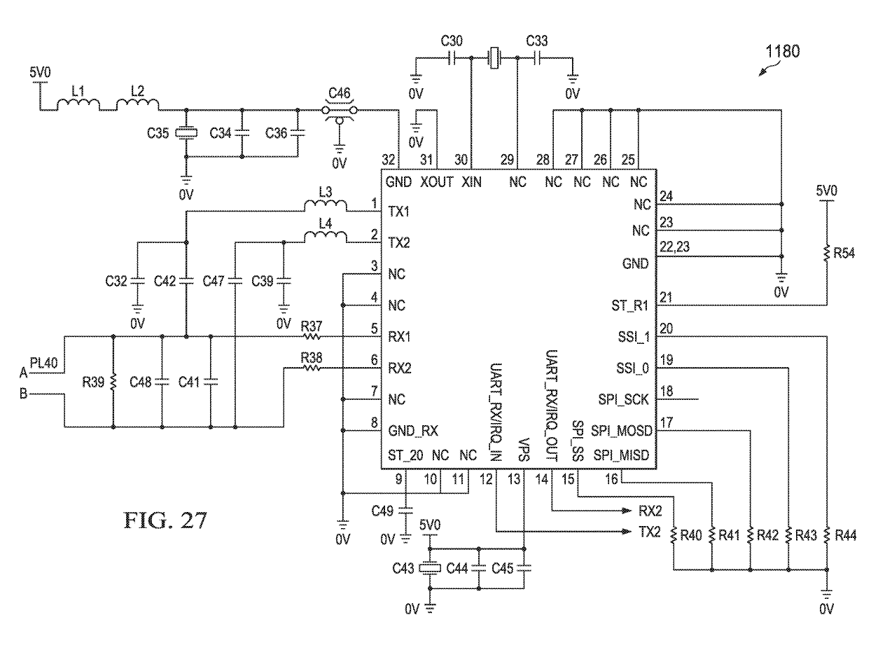

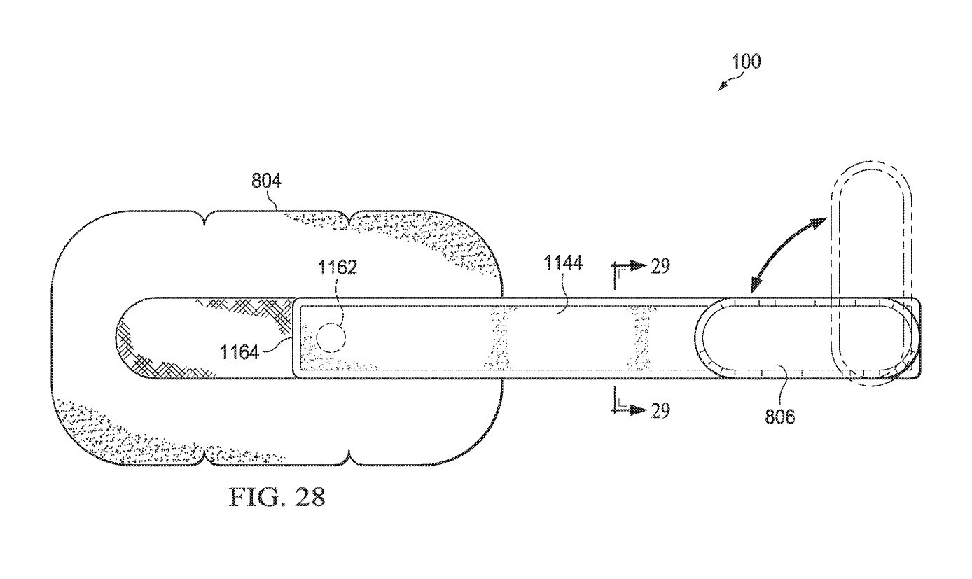

FIGS. 26-28 illustrate another example embodiment of the system 100. In this example configuration, a bridge 1144 couples the dressing 804 to the pump assembly 806. The bridge 1144 may be longitudinally elongated in some embodiments, such as illustrated in the example of FIG. 26. The internal components of the bridge 1144 may be similar or analogous to components disclosed in U.S. patent application Ser. No. 15/356,063 entitled “Medical System and Dressing for Use Under Compression” which was filed on Nov. 18, 1016, and is incorporated by reference herein. In the example of FIGS. 26-28, the bridge 1144 includes a coated top cover 1146 and bottom cover 1148, and at least two wicking layers 1150 between the top cover 1146 and the bottom cover 1148. Polyurethane may be a suitable material for some embodiments of the top cover 1146, the bottom cover 1148, or both. The wicking layers 1150 may be joined together at their peripheries and define internal elongated passageways. An absorbent layer 1152 may be optionally included between at least a pair of the wicking layers 1150. An adhesive ring 1160 may be aligned with an inlet hole 1162 adjacent a proximal end 1164, to attach the bridge 1144 to the laterally enlarged outer cover of the dressing 804. In some embodiments, the bridge 1144 may include the inlet 862, the filter 846, and the adhesive ring 848. As illustrated in the example of FIG. 28, the inlet 862 may include the interlocking wings 838, which can removably couple to the interlocking ledges 892 of the pump assembly 806. As illustrated in the example of FIG. 26, the pump assembly 806 may have a lateral width that is substantially the same as a width of the bridge 1144, for alignment therewith in the longitudinal direction when fully attached thereto.

The systems, apparatuses, and methods described herein may provide significant advantages. For example, the pump assembly 806 can be removeably connected to the dressing 804 in a fast and tool-free direct connected manner without an intervening flexible tube or hose. The smooth exterior surface and self-contained nature of the present pump assembly, with the inductive power receptacle, allows for easy user cleaning of the device while remaining externally sealed but for the single air and liquid inlet opening. The dressing can be sterilized without impacting the removed pump assembly, such as by use of ethylene oxide and/or heat. It may also be beneficial that the dressing can be disposed of as medical waste separately from the pump assembly, which may be recycled or treated as less expensive waste. As another advantage, a single pump assembly can be sequentially used for multiple dressings, especially if the first dressing becomes saturated with wound liquids.

Additionally, in some embodiments the antenna 1186 and the antenna 1194 may act to align the transmitter and receiver prior to charging in some embodiments. The inductive charging system can be advantageous and may not require any exposed apertures in the pump cover, which are otherwise prone to contamination or water entry, to allow for receiving an external wall plug and wire. The inductive charging system may also be advantageous by not requiring a more complicated, expensive and removable access cover and connectors for allowing replacement of the pump battery.

While shown in a few illustrative embodiments, a person having ordinary skill in the art will recognize that the systems, apparatuses, and methods described herein are susceptible to various changes and modifications that fall within the scope of the appended claims. Moreover, descriptions of various alternatives using terms such as “or” do not require mutual exclusivity unless clearly required by the context, and the indefinite articles “a” or “an” do not limit the subject to a single instance unless clearly required by the context. Components may be also be combined or eliminated in various configurations for purposes of sale, manufacture, assembly, or use. For example, in some configurations the dressing 804 and the pump assembly 806 may be separated for manufacture or sale.

The appended claims set forth novel and inventive aspects of the subject matter described above, but the claims may also encompass additional subject matter not specifically recited in detail. For example, certain features, elements, or aspects may be omitted from the claims if not necessary to distinguish the novel and inventive features from what is already known to a person having ordinary skill in the art. Features, elements, and aspects described in the context of some embodiments may also be omitted, combined, or replaced by alternative features serving the same, equivalent, or similar purpose without departing from the scope of the invention defined by the appended claims.

The invention claimed is:

1. A system for treating a tissue site with reduced pressure, the system comprising:
a dressing comprising:
an absorbent layer;
a manifold adjacent to the absorbent layer;
a dressing cover enclosing the absorbent layer and the manifold; and
a fluid outlet;
a pump assembly comprising:
a pump; and
a fluid inlet coupled to the pump;
wherein the fluid inlet is configured to be removeably and directly connectable to the fluid outlet by interlocking formations.

2. The system of claim 1, wherein the pump assembly further comprises:
an inner cover and an outer cover joined together at a periphery to enclose the pump;
a cushion between the inner cover and the outer cover and adjacent to the pump; and
an aperture in the inner cover, the fluid inlet being accessible through the aperture.

3. The system of claim 2, wherein:
the pump assembly further comprises an electrical circuit connected to the pump and a battery connected to the electrical circuit;
the inner cover is located between the electrical circuit and the dressing cover;
a periphery of the inner cover is secured to a periphery of the outer cover; and
the cushion is located between: the outer cover and an area defined by the electrical circuit and the battery.

4. The system of claim 1, further comprising:
a battery powering the pump; and
an inductive charging assembly adapted to electrically charge the battery.

5. The system of claim 4, wherein the inductive charging assembly comprises:
a charging stand electrically configured to be connected to a wall outlet;
a receiver electrically connected to the battery; and
magnets removeably coupling the charging stand to the receiver and also configured to inductively transfer a battery charge.

6. The system of claim 1, wherein the interlocking formations comprise at least two wings outwardly extending from at least one of the fluid outlet and the fluid inlet.

7. The system of claim 6, wherein the interlocking formations comprise ledges between which are receptacles configured to receive the wings if at least one of the interlocking formations is moved to secure together the dressing and the pump assembly.

8. The system of claim 1, wherein the pump assembly comprises:
a printed circuit board connected to the pump;
a battery flexibly connected to the printed circuit board and having at least one surface substantially coplanar with the printed circuit board; and
a pump cover substantially enclosing the pump, the battery, and the printed circuit board.

9. The system of claim 1, wherein the pump assembly further comprises a breathable cushion surrounding at least a majority of a periphery of the pump.

10. The system of claim 1, wherein the fluid inlet and the fluid outlet are engageable and disengageable by rotating at least one of the interlocking formations.

11. The system of claim 1, wherein the dressing and the pump assembly are secured together by only the interlocking formations of the fluid inlet and the fluid outlet.

12. The system of claim 1, wherein at least one of the fluid inlet and the fluid outlet are tapered.

13. The system of claim 1, wherein the fluid inlet is directly connected to the fluid outlet that extends through an aperture in the dressing cover above the manifold, and the pump assembly further comprises a pump cover adjacent to the dressing cover when the fluid inlet and the fluid outlet are connected together.

14. The system of claim 1, further comprises an elongated bridge coupling the fluid inlet of the pump assembly to the dressing, wherein the fluid outlet is disposed adjacent to an end of the bridge furthest away from the manifold, and the bridge further comprises outer manifold layers coupled together with absorbent and wicking layers within a hollow interior between the outer manifold layers.

15. A system for providing reduced-pressure therapy, the system comprising:
an elongated dressing comprising:
an absorbent layer;
a manifold;
a dressing cover enclosing the absorbent layer and the manifold;
a first aperture in the dressing cover; and
an outlet coupled to the manifold and being accessible through the first aperture;
an elongated pump pouch comprising:
a pump;
an elongated and flexible cushion;
a pump cover enclosing the pump and the cushion;
a second aperture in the pump cover;
an inlet coupled to the pump and being accessible through the second aperture; and
the dressing being removeably coupled to the pump pouch solely by the inlet being attached to the outlet, and the dressing cover and the pump cover contact each other while the inlet and the outlet are attached together.

16. The system of claim 15, further comprising interlocking formations coupling together the inlet and the outlet through a rotational motion.

17. The system of claim 16, wherein the interlocking formations comprise:
wings outwardly extending from one of the outlet and the inlet; and
receptacles configured to receiving and engaging the wings.

18. The system of claim 15, further comprising:
a battery powering the pump; and
an inductive charger configured to electrically charging the battery.

19. The system of claim 18, wherein the inductive charger comprises:
a charging stand configured to be electrically connected to a wall outlet;
a receiver electrically connected to the battery; and
magnets removeably coupling the stand to the receiver and configured to transmit an inductive charge.

20. The system of claim 15, further comprising:
a printed circuit board located within the pump cover and being connected to the pump; and
a battery located within the pump cover and being connected to the printed circuit board.

21. The system of claim 15, wherein:
elongation directions of the dressing and the pump pouch are substantially parallel when coupled together;
the pump pouch includes an elongation dimension at least twice a width dimension;
the width dimension is at least five times greater than a thickness dimension; and
the inlet is adjacent to an end of the pump cover.

22. The system of claim 15, wherein the dressing further comprises a hydrophobic filter, and the pump is a piezoelectric pump.

23. The system of claim 15, wherein the inlet and outlet directly couple the manifold to the pump without any elongated conduits therebetween.

24. A reduced-pressure treatment system comprising:
a pump;
an electrical circuit connected to the pump;
a battery connected to the electrical circuit;
a flexible cushion;
a flexible pump cover substantially surrounding the pump, the electrical circuit, the battery, and the cushion;
the cushion being located between the electrical circuit and a portion of the pump cover; and
a dressing connection fluidly coupled to the pump.

25. The system of claim 24, further comprising:
a dressing including a rigid outlet; and
interlocking formations removeably coupling together the dressing connection and the outlet through a rotational movement.

26. The system of claim 25, wherein the interlocking formations comprise:
wings outwardly extending from one of the connection and the outlet; and
receptacles located between ledges for operably receiving and engaging the wings.

27. The system of claim 24, further comprising an inductive charger configured to electrically charge the battery.

28. The system of claim 27, wherein the inductive charger comprises:
a charging stand configured to be electrically connected to a wall outlet;
a receiver electrically connected to the battery; and
magnets removeably coupling the stand to the receiver and configured to transmit an inductive charge.

29. The system of claim 24, wherein:
the battery is substantially coplanar with the electrical circuit, which is on a circuit board; and
the battery is flexibly connected to the circuit board.

30. The system of claim 29, wherein the cushion is breathable foam.

31. The system of claim 24, wherein the cushion surrounds a periphery of the pump and is elongated parallel to a centerline through the pump, circuit and battery.

32. The system of claim 24, wherein the pump is a piezoelectric pump, and further comprising a dressing including an absorbent layer, a manifold and a flexible cover.

33. The system of claim 24, further comprising an elongated bridge coupling an outlet of a dressing to the dressing connection, the bridge comprising a polymeric cover and absorbent layers.

34. A system for treating a tissue site, the system comprising:
a pump configured to provide reduced pressure;
a flexible cushion substantially surrounding at least a majority of a periphery of the pump;
a flexible cover substantially surrounding the pump and the cushion; and
a dressing connection fluidically coupled to the pump.

35. The system of claim 34, further comprising:
a dressing including a rigid outlet; and
interlocking formations removeably coupling together the dressing connection and the outlet through a rotational movement.

36. The system of claim 35, wherein the interlocking formations comprise:
wings outwardly extending from one of the dressing connection and the outlet; and
receptacles for operably receiving and engaging the wings.

37. The system of claim 34, further comprising:
a battery located within the cover for operably powering the pump; and
an inductive charger for electrically charging the battery.

38. The system of claim 37, wherein the inductive charger comprises:
a charging stand configured to be electrically connected to a wall outlet;
a receiver electrically connected to the battery; and
magnets removeably coupling the stand to the receiver and configured to transmit an inductive charge.

39. The system of claim 34, wherein the pump is a piezoelectric pump, and further comprising a dressing including an absorbent layer, a manifold and a flexible cover.

40. The system of claim 34, further comprising an electrical circuit and an offset battery located within the cover, wherein the cushion is breathable foam and is elongated to cover the circuit and the battery.

41. The system of claim 34, further comprising an elongated bridge coupled to the dressing connection, the bridge comprising a polymeric cover and absorbent layers.

42. A reduced-pressure wound treatment system comprising:
a pump;
an electrical circuit board connected to the pump;
a battery configured to power the pump, the battery flexibly coupled to and longitudinally offset from the electrical circuit board;
a flexible cover surrounding the pump, electrical circuit board, and the battery; and
a dressing connection fluidically coupled to the pump.

43. The system of claim 42, further comprising:
a dressing including a rigid outlet; and
interlocking formations removeably coupling together the dressing connection and the outlet through a rotational movement.

44. The system of claim 42, further comprising an inductive charger electrically charging the battery.

45. The system of claim 42, further comprising a breathable and flexible cushion located around a periphery of the pump and between the electrical circuit board and a portion of the cover.

46. The system of claim 42, wherein the battery has a substantially polygonal periphery and flat outer and inner surfaces, the electrical circuit board is located between the battery and the pump in an offset configuration, and a surface of the battery and a surface of the electrical circuit board are substantially co-planar.

47. A reduced-pressure wound treatment system comprising:
a dressing;
a pump assembly coupled to the dressing, the pump assembly comprising:
a pump;
a battery operably powering the pump;
a cover substantially surrounding the pump and battery; and
an inductive charger operable to electrically charge the battery while the battery remains within the cover.

48. The system of claim 47, wherein the charger includes a support base with an inductive antenna, an electrical circuit connected to the antenna and a wall outlet plug connected to the circuit.

49. The system of claim 48, wherein the antenna is a magnet.

50. The system of claim 48, further comprising a receiver attached to the pump assembly, and a magnet removeably coupling the base to the receiver.

51. The system of claim 47, wherein the charger includes a ring magnet with an open center.

52. The system of claim 47, wherein the charger includes a disc magnet with a sold metallic center.

53. The system of claim 47, wherein the charger includes:
a charging stand configured to be electrically connected to a wall outlet;
a receiver electrically connected to the battery; and
magnets removeably coupling the stand to the receiver and operable to inductively charge the battery.

54. The system of claim 47, wherein the charger comprises a receiver and a substantially semispherical base, the receiver being electrically attached to the battery and the base being removeable, and the base configured to inductively transmit power of 15 watts or less to the receiver to charge the battery.

55. The system of claim 47, wherein the pump is a piezoelectric pump, and the dressing comprises an absorbent layer, a manifold and a flexible cover.

56. The system of claim 47, further comprising a printed circuit board connecting and being located between the battery and the pump, at least a surface of the battery being substantially coplanar with the printed circuit board, and the battery being flexibly connected to the printed circuit board.

57. A method for electrically charging a reduced-pressure treatment system, the method comprising:
placing a pump assembly, comprising a pump and a battery within a cover, adjacent to an electrical charger;
electrically charging the battery via inductance from the charger without removing the battery from the pump assembly; and
coupling the pump assembly to a wound dressing.

58. The method of claim 57, further comprising transmitting a charge via a magnet antenna.

59. The method of claim 57, further comprising:
removeably aligning a support base of the charger to a receiver, the receiver being electrically attached to the pump assembly;
connecting a plug of the base to an electrical wall outlet; and
the cover being flexible.

60. A method of using a reduced-pressure treatment system, the method comprising:
aligning an inlet of a pump assembly to an outlet of a reduced-pressure dressing;
inserting projections from one of the inlet and the outlet into openings of the other of the inlet and the outlet; and
rotating the projections relative to the openings in order to interlock together the inlet and the outlet.

61. The method of claim 60, wherein the pump assembly is solely retained to the reduced-pressure dressing by the interlocked inlet and outlet.

62. The method of claim 60, wherein the reduced-pressure dressing includes an elongated and flexible bridge including a cover and absorbent layers therein, the outlet being located adjacent a distal end of the bridge, aligning the inlet of the pump assembly with the outlet of the bridge, and engaging the projections with ledges during the rotating.
